SEQUENCE LISTING (8 sequences)

SEQ ID NO: 1 - DNA, 2016 bp, Homo sapiens
  human RIPK1
atgcaaccagacatgtccttgaatgtcattaagatgaaatccagtgacttcctggagagtgcagaactggacagcggaggctttgggaaggtgtctctgtgtttccacagaacccagggactcatgatcatgaaaacagtgtacaaggggcccaactgcattgagcacaacgaggccctcttggaggaggcgaagatgatgaacagactgagacacagccgggtggtgaagctcctgggcgtcatcatagaggaagggaagtactccctggtgatggagtacatggagaagggcaacctgatgcacgtgctgaaagccgagatgagtactccgctttctgtaaaaggaaggataattttggaaatcattgaaggaatgtgctacttacatggaaaaggcgtgatacacaaggacctgaagcctgaaaatatccttgttgataatgacttccacattaagatcgcagacctcggccttgcctcctttaagatgtggagcaaactgaataatgaagagcacaatgagctgagggaagtggacggcaccgctaagaagaatggcggcaccctctactacatggcgcccgagcacctgaatgacgtcaacgcaaagcccacagagaagtcggatgtgtacagctttgctgtagtactctgggcgatatttgcaaataaggagccatatgaaaatgctatctgtgagcagcagttgataatgtgcataaaatctgggaacaggccagatgtggatgacatcactgagtactgcccaagagaaattatcagtctcatgaagctctgctgggaagcgaatccggaagctcggccgacatttcctggcattgaagaaaaatttaggcctttttatttaagtcaattagaagaaagtgtagaagaggacgtgaagagtttaaagaaagagtattcaaacgaaaatgcagttgtgaagagaatgcagtctcttcaacttgattgtgtggcagtaccttcaagccggtcaaattcagccacagaacagcctggttcactgcacagttcccagggacttgggatgggtcctgtggaggagtcctggtttgctccttccctggagcacccacaagaagagaatgagcccagcctgcagagtaaactccaagacgaagccaactaccatctttatggcagccgcatggacaggcagacgaaacagcagcccagacagaatgtggcttacaacagagaggaggaaaggagacgcagggtctcccatgacccttttgcacagcaaagaccttacgagaattttcagaatacagagggaaaaggcactgcttattccagtgcagccagtcatggtaatgcagtgcaccagccctcagggctcaccagccaacctcaagtactgtatcagaacaatggattatatagctcacatggctttggaacaagaccactggatccaggaacagcaggtcccagagtttggtacaggccaattccaagtcatatgcctagtctgcataatatcccagtgcctgagaccaactatctaggaaatacacccaccatgccattcagctccttgccaccaacagatgaatctataaaatataccatatacaatagtactggcattcagattggagcctacaattatatggagattggtgggacgagttcatcactactagacagcacaaatacgaacttcaaagaagagccagctgctaagtaccaagctatctttgataataccactagtctgacggataaacacctggacccaatcagggaaaatctgggaaagcactggaaaaactgtgcccgtaaactgggcttcacacagtctcagattgatgaaattgaccatgactatgagcgagatggactgaaagaaaaggtttaccagatgctccaaaagtgggtgatgagggaaggcataaagggagccacggtggggaagctggcccaggcgctccaccagtgttccaggatcgaccttctgagcagcttgatttacgtcagccagaactaa

SEQ ID NO: 2 - protein, 671 aa, Homo sapiens
  human RIPK1
MQPDMSLNVIKMKSSDFLESAELDSGGFGKVSLCFHRTQGLMIMKTVYKGPNCIEHNEALLEEAKMMNRLRHSRVVKLLGVIIEEGKYSLVMEYMEKGNLMHVLKAEMSTPLSVKGRIILEIIEGMCYLHGKGVIHKDLKPENILVDNDFHIKIADLGLASFKMWSKLNNEEHNELREVDGTAKKNGGTLYYMAPEHLNDVNAKPTEKSDVYSFAVVLWAIFANKEPYENAICEQQLIMCIKSGNRPDVDDITEYCPREIISLMKLCWEANPEARPTFPGIEEKFRPFYLSQLEESVEEDVKSLKKEYSNENAVVKRMQSLQLDCVAVPSSRSNSATEQPGSLHSSQGLGMGPVEESWFAPSLEHPQEENEPSLQSKLQDEANYHLYGSRMDRQTKQQPRQNVAYNREEERRRRVSHDPFAQQRPYENFQNTEGKGTAYSSAASHGNAVHQPSGLTSQPQVLYQNNGLYSSHGFGTRPLDPGTAGPRVWYRPIPSHMPSLHNIPVPETNYLGNTPTMPFSSLPPTDESIKYTIYNSTGIQIGAYNYMEIGGTSSSLLDSTNTNFKEEPAAKYQAIFDNTTSLTDKHLDPIRENLGKHWKNCARKLGFTQSQIDEIDHDYERDGLKEKVYQMLQKWVMREGIKGATVGKLAQALHQCSRIDLLSSLIYVSQN

SEQ ID NO: 3 - DNA, 1557 bp, Homo sapiens
  human RIPK3
atgtcgtgcgtcaagttatggcccagcggtgcccccgcccccttggtgtccatcgaggaactggagaaccaggagctcgtcggcaaaggcgggttcggcacagtgttccgggcgcaacataggaagtggggctacgatgtggcggtcaagatcgtaaactcgaaggcgatatccagggaggtcaaggccatggcaagtctggataacgaattcgtgctgcgcctagaaggggttatcgagaaggtgaactgggaccaagatcccaagccggctctggtgactaaattcatggagaacggctccttgtcggggctgctgcagtcccagtgccctcggccctggccgctcctttgccgcctgctgaaagaagtggtgcttgggatgttttacctgcacgaccagaacccggtgctcctgcaccgggacctcaagccatccaacgtcctgctggacccagagctgcacgtcaagctggcagattttggcctgtccacatttcagggaggctcacagtcagggacagggtccggggagccagggggcaccctgggctacttggccccagaactgtttgttaacgtaaaccggaaggcctccacagccagtgacgtctacagcttcgggatcctaatgtgggcagtgcttgctggaagagaagttgagttgccaaccgaaccatcactcgtgtacgaagcagtgtgcaacaggcagaaccggccttcattggctgagctgccccaagccgggcctgagactcccggcttagaaggactgaaggagctaatgcagctctgctggagcagtgagcccaaggacagaccctccttccaggaatgcctaccaaaaactgatgaagtcttccagatggtggagaacaatatgaatgctgctgtctccacggtaaaggatttcctgtctcagctcaggagcagcaataggagattttctatcccagagtcaggccaaggagggacagaaatggatggctttaggagaaccatagaaaaccagcactctcgtaatgatgtcatggtttctgagtggctaaacaaactgaatctagaggagcctcccagctctgttcctaaaaaatgcccgagccttaccaagaggagcagggcacaagaggagcaggttccacaagcctggacagcaggcacatcttcagattcgatggcccaacctccccagactccagagacctcaactttcagaaaccagatgcccagccctacctcaactggaacaccaagtcctggaccccgagggaatcagggggctgagagacaaggcatgaactggtcctgcaggaccccggagccaaatccagtaacagggcgaccgctcgttaacatatacaactgctctggggtgcaagttggagacaacaactacttgactatgcaacagacaactgccttgcccacatggggcttggcaccttcgggcaaggggaggggcttgcagcaccccccaccagtaggttcgcaagaaggccctaaagatcctgaagcctggagcaggccacagggttggtataatcatagcgggaaataa

SEQ ID NO: 4 - protein, 518 aa, Homo sapiens
  human RIPK3
MSCVKLWPSGAPAPLVSIEELENQELVGKGGFGTVFRAQHRKWGYDVAVKIVNSKAISREVKAMASLDNEFVLRLEGVIEKVNWDQDPKPALVTKFMENGSLSGLLQSQCPRPWPLLCRLLKEVVLGMFYLHDQNPVLLHRDLKPSNVLLDPELHVKLADFGLSTFQGGSQSGTGSGEPGGTLGYLAPELFVNVNRKASTASDVYSFGILMWAVLAGREVELPTEPSLVYEAVCNRQNRPSLAELPQAGPETPGLEGLKELMQLCWSSEPKDRPSFQECLPKTDEVFQMVENNMNAAVSTVKDFLSQLRSSNRRFSIPESGQGGTEMDGFRRTIENQHSRNDVMVSEWLNKLNLEEPPSSVPKKCPSLTKRSRAQEEQVPQAWTAGTSSDSMAQPPQTPETSTFRNQMPSPTSTGTPSPGPRGNQGAERQGMNWSCRTPEPNPVTGRPLVNIYNCSGVQVGDNNYLTMQQTTALPTWGLAPSGKGRGLQHPPPVGSQEGPKDPEAWSRPQGWYNHSGK

SEQ ID NO: 5 - DNA, 1416 bp, Homo sapiens
  human MLKL ISOFORM 1
atggaaaatttgaagcatattatcacccttggccaggtcatccacaaacggtgtgaagagatgaaatactgcaagaaacagtgccggcgcctgggccaccgcgtcctcggcctgatcaagcctctggagatgctccaggaccaaggaaagaggagcgtgccctctgagaagttaaccacagccatgaaccgcttcaaggctgccctggaggaggctaatggggagatagaaaagttcagcaatagatccaatatctgcaggtttctaacagcaagccaggacaaaatactcttcaaggacgtgaacaggaagctgagtgatgtctggaaggagctctcgctgttacttcaggttgagcaacgcatgcctgtttcacccataagccaaggagcgtcctgggcacaggaagatcagcaggatgcagacgaagacaggcgagctttccagatgctaagaagagataatgaaaaaatagaagcttcactgagacgattagaaatcaacatgaaagaaatcaaggaaactttgaggcagtatttaccaccaaaatgcatgcaggagatcccgcaagagcaaatcaaggagatcaagaaggagcagctttcaggatccccgtggattctgctaagggaaaatgaagtcagcacactttataaaggagaataccacagagctccagtggccataaaagtattcaaaaaactccaggctggcagcattgcaatagtgaggcagactttcaataaggagatcaaaaccatgaagaaattcgaatctcccaacatcctgcgtatatttgggatttgcattgatgaaacagtgactccgcctcaattctccattgtcatggagtactgtgaactcgggaccctgagggagctgttggatagggaaaaagacctcacacttggcaagcgcatggtcctagtcctgggggcagcccgaggcctataccggctacaccattcagaagcacctgaactccacggaaaaatcagaagctcaaacttcctggtaactcaaggctaccaagtgaagcttgcaggatttgagttgaggaaaacacagacttccatgagtttgggaactacgagagaaaagacagacagagtcaaatctacagcatatctctcacctcaggaactggaagatgtattttatcaatatgatgtaaagtctgaaatatacagctttggaatcgtcctctgggaaatcgccactggagatatcccgtttcaaggctgtaattctgagaagatccgcaagctggtggctgtgaagcggcagcaggagccactgggtgaagactgcccttcagagctgcgggagatcattgatgagtgccgggcccatgatccctctgtgcggccctctgtggatgaaatcttaaagaaactctccaccttttctaagtag

SEQ ID NO: 6 - protein, 471 aa, Homo sapiens
  human MLKL ISOFORM 1
MENLKHIITLGQVIHKRCEEMKYCKKQCRRLGHRVLGLIKPLEMLQDQGKRSVPSEKLTTAMNRFKAALEEANGEIEKFSNRSNICRFLTASQDKILFKDVNRKLSDVWKELSLLLQVEQRMPVSPISQGASWAQEDQQDADEDRRAFQMLRRDNEKIEASLRRLEINMKEIKETLRQYLPPKCMQEIPQEQIKEIKKEQLSGSPWILLRENEVSTLYKGEYHRAPVAIKVFKKLQAGSIAIVRQTFNKEIKTMKKFESPNILRIFGICIDETVTPPQFSIVMEYCELGTLRELLDREKDLTLGKRMVLVLGAARGLYRLHHSEAPELHGKIRSSNFLVTQGYQVKLAGFELRKTQTSMSLGTTREKTDRVKSTAYLSPQELEDVFYQYDVKSEIYSFGIVLWEIATGDIPFQGCNSEKIRKLVAVKRQQEPLGEDCPSELREIIDECRAHDPSVRPSVDEILKKLSTFSK

SEQ ID NO: 7 - DNA, 792 bp, Homo sapiens
  human MLKL ISOFORM 2
atggaaaatttgaagcatattatcacccttggccaggtcatccacaaacggtgtgaagagatgaaatactgcaagaaacagtgccggcgcctgggccaccgcgtcctcggcctgatcaagcctctggagatgctccaggaccaaggaaagaggagcgtgccctctgagaagttaaccacagccatgaaccgcttcaaggctgccctggaggaggctaatggggagatagaaaagttcagcaatagatccaatatctgcaggtttctaacagcaagccaggacaaaatactcttcaaggacgtgaacaggaagctgagtgatgtctggaaggagctctcgctgttacttcaggttgagcaacgcatgcctgtttcacccataagccaaggagcgtcctgggcacaggaagatcagcaggatgcagacgaagacaggcgagctttccagatgctaagaagagataatgaaaaaatagaagcttcactgagacgattagaaatcaacatgaaagaaatcaaggaaactttgaggcagtctttggaatcgtcctctgggaaatcgccactggagatatcccgtttcaaggtgaagaatgtgaagactggctcagccagtggctgtaattctgagaagatccgcaagctggtggctgtgaagcggcagcaggagccactgggtgaagactgcccttcagagctgcgggagatcattgatgagtgccgggcccatgatccctctgtgcggccctctgtggatgaaatcttaaagaaactctccaccttttctaagtag

SEQ ID NO: 8 - protein, 262 aa, Homo sapiens
  human MLKL ISOFORM 2
MENLKHIITLGQVIHKRCEEMKYCKKQCRRLGHRVLGLIKPLEMLQDQGKRSVPSEKLTTAMNRFKAALEEANGEIEKFSNRSNICRFLTASQDKILFKDVNRKLSDVWKELSLLLQVEQRMPVSPISQGASWAQEDQQDADEDRRAFQMLRRDNEKIEASLRRLEINMKEIKETLRQSLESSSGKSPLEISRFKVKNVKTGSASGCNSEKIRKLVAVKRQQEPLGEDCPSELREIIDECRAHDPSVRPSVDEILKKLSTFS

(19) United States
(12) Patent Application Publication
(10) Pub. No.: US 2017/0226514 A1
(43) Pub. Date: Aug. 10, 2017

(54) PREVENTION OF TUMOUR METASTASIS BY INHIBITION OF NECROPTOSIS

(71) Applicant: MAX-PLANCK-GESELLSCHAFT ZUR FÖRDERUNG DER WISSENSCHAFTEN E.V., München (DE)

(72) Inventors: Stefan OFFERMANNS, Bad Nauheim (DE); Boris STRILIC, Bad Nauheim (DE)

(21) Appl. No.: 15/503,583

(22) PCT Filed: Aug. 11, 2015
(86) PCT No.: PCT/EP2015/068495

§ 371 Date: Feb. 13, 2017

(30) Foreign Application Priority Data
Aug. 13, 2014 (EP) 14180878.2

(51) Int. Cl. C12N 15/115; C07K 2/00; G01N 33/50; C07D 417/06; C07D 403/06; C07D 409/12; C07K 16/40; C12N 15/113

(52) U.S. Cl. CPC C12N 15/115; C07K 16/40; C07K 2/00; C12N 15/1137; C07D 417/06; C07D 403/06; C07D 409/12; G01N 33/5011; C07K 2318/00; C12N 2310/14; C12N 2310/531; C12N 2310/141; C12N 2310/16; C12N 2310/11; C07K 2317/76; C12N 2310/12

(57) ABSTRACT

The present invention relates to an inhibitor of necroptosis for use in preventing the metastasis of tumours. Further, the present invention relates to a method of preventing the metastasis of tumours by inhibiting necroptosis and to a method for modulating the transmigration of metastasising tumour cells through endothelium by modulating necroptosis as well as to an in-vitro method of identifying an inhibitor of necroptosis suitable as a lead compound and/or as a medicament for the prevention of tumour metastasis. Moreover, the present invention also relates to a method of identifying necroptotic and necrotic cells.

The present invention relates to an inhibitor of necroptosis for use in preventing the metastasis of tumours. Further, the present invention relates to a method of preventing the metastasis of tumours by inhibiting necroptosis and to a method for modulating the transmigration of metastasising tumour cells through endothelium by modulating necroptosis as well as to an in-vitro method of identifying an inhibitor of necroptosis suitable as a lead compound and/or as a medicament for the prevention of tumour metastasis. Moreover, the present invention also relates to a method of identifying necroptotic and necrotic cells.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

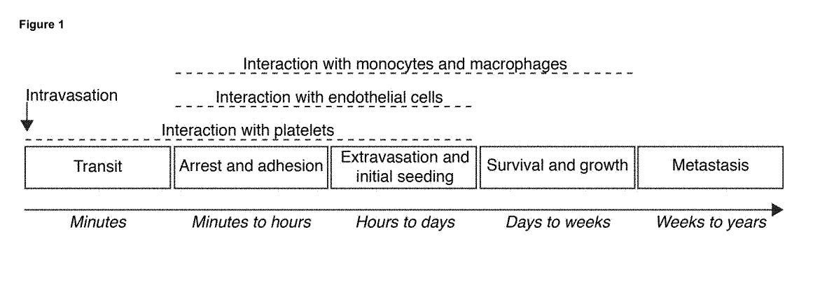

Tumour cell metastasis is the primary cause of mortality in cancer patients. The process of metastasis is a timed sequence of events that begins with the escape of tumour cells from the primary tumour. This is followed by the intravasation into the circulatory system. Once in the blood stream, the tumour cells' metastatic potential depends on their interplay with blood cells and endothelial cells (FIG. 1). The metastatic potential of tumour cells largely depends on a rapid and efficient way to extravasate into the surrounding tissue. Paracrine signals released from tumour cells or tumour cell-activated immune cells induce local activation of endothelial cells which results in an exposure of adhesion molecules that enables tumour cells to firmly adhere to the endothelium (Joyce and Pollard, 2009) (FIG. 1). Within the next hours, tumour cells further modulate the endothelium to induce an opening of the endothelial barrier to facilitate extravasation. These modulations include for example changes in endothelial junctions and cytoskeletal changes (Labelle and Hynes, 2012; Mierke, 2008). Once extravasated into the parenchyma of a distant organ, the further metastatic potential of tumour cells depends on their ability to survive, adhere and to further proliferate at sites of extravasation.

The metastatic potential of tumour cells is largely modulated by immune cells and endothelial cells. The latter are of particular interest as they are the primary site where extravasation of tumour cells occurs and since they are thought to actively regulate metastasis formation. For example, tumour cell extravasation includes active rearrangement of endothelial cell-cell junctions as well as cytoskeletal modulations.

The present dogma of tumour cell extravasation suggests that, similar to leukocytes, tumour cells transmigrate the endothelium without significantly disrupting the endothelial monolayer by inducing endothelial cell retraction via cellular rearrangements (Reymond et al., 2013). There are only few reports suggesting alternative mechanisms such as induction of apoptosis in the endothelial cells by direct contact with the tumour cells (Heyder et al., 2002; Kebers et al., 1998; Lin et al., 2011).

Besides the well characterised apoptotic cell death, other forms of cell death such as necrosis exist. While apoptosis describes a form of ‘programmed cell death’ that is regulated by a variety of cellular signalling pathways, necrosis describes an ‘accidental’ form of cell death that is induced by toxic insults or physical damage. Apoptosis can be induced by activation of surface receptors and is intracellularly transduced by caspases. Blockade or inhibition of caspases can prevent apoptosis.

In contrast to apoptosis, necrosis is induced by external factors such as extreme cold, heat or hypoxia, and no signalling molecules are involved in the regulation of necrotic cell death. Morphologically, apoptosis coincides with nuclear condensation and fragmentation, cleavage of chromosomal DNA and packaging of the deceased cell into apoptotic bodies without plasma membrane breakdown. Only at later stages of apoptosis, plasma membrane breakdown can occur (i.e. late apoptosis). In contrast, necrotic cells are characterised morphologically by cellular swelling and breakdown of the plasma membrane and only minor changes in nuclear morphology without organised chromatin condensation and fragmentation.

More recently, it was described that programmable cell death can also occur in the absence of apoptotic but presence of necrotic features and is termed regulated necrosis or ‘necroptosis’ (Linkermann and Green, 2014; Murphy and Silke, 2014). Necroptosis follows intracellular signalling pathways which are not executed or positively regulated by caspases. Instead, molecules such as receptor interacting protein kinase 1 (RIPK1), RIPK3 and mixed lineage kinase like domain (MLKL) were identified as key regulators of necroptosis (Linkermann and Green, 2014; Murphy and Silke, 2014).

So far, necroptotic signalling was studied mainly in in-vitro systems due to limiting tools to study its relevance in vivo under physiological or pathophysiological conditions. Perhaps the best evidenced role for necroptosis in vivo is in the regulation of viral infection (Cho et al., 2009). Further in-vivo evidence comes from studies in the context of myocardial infarction and stroke (Smith et al., 2007), atherosclerosis (Lin et al., 2013), ischemia reperfusion injury (Linkermann et al., 2012) and some others. However, no study was able to directly show necroptotic cell death in vivo. Instead, genetic models are commonly used to indirectly study necroptosis in vivo with animals harbouring single or multi gene mutations of the necroptotic pathway (Dillon et al., 2012; Dillon et al., 2014).

The discrimination between apoptotic and necroptotic cell death is often based on the analysis of morphological features by electron microscopy. However, this method is extremely laborious and not feasible for the analysis of large amounts of samples or to study the systemic relevance in an organism. Alternative methods were established to discriminate these two forms of cell death. For example, exposure of phosphatidylserines on the plasma membrane is often used as a marker for apoptotic cells and can be visualised by staining for Annexin V. However, necroptotic cells also expose phosphatidylserines (Sawai and Domae, 2011). On the other hand, membrane-impermeable fluorescent dyes that bind to DNA are often used to identify necroptotic cells since apoptotic and living cells have intact membranes and stain negative. However, as mentioned above, late apoptotic cells lose their membrane integrity and thus also stain positive for these dyes. Taken together, besides electron microscopy, there is no available method to reliably distinguish apoptotic from necroptotic cell death.

To overcome this shortcoming, researchers tend to use indirect approaches to discriminate between apoptotic and necroptotic cell death. For example, general cell viability (e.g. by measuring ATP levels, Trypan blue exclusion, etc.) is determined in the presence of apoptosis- and/or necroptosis-inhibiting substances or gene knockdowns of molecules of the respective cell death pathway. However, these methods are limited to in-vitro systems and no assay is available as of today that can be used to reliably discriminate apoptotic and necrotic/necroptotic cells in vivo. Moreover, inhibition or knockdown/knockout of components required for one form of cell death can potentially artificially influence the type of death and thus lead to incorrect results regarding the actual type of death occurring under physiological conditions.

Although many advances have been made in the treatment of cancer, tumour cell metastasis remains a major cause of mortality in cancer patients. Thus, alternative and/or improved treatments preventing the metastasis of tumours are needed.

Further, due to the above mentioned drawbacks associated with the current methods of determining necroptotic cell death, alternative and/or improved methods for measuring necroptotic cell death are needed.

The technical problem underlying the present invention was to identify alternative and/or improved means and methods to prevent tumour metastasis and/or to identify alternative and/or improved methods for detecting necroptotic cells.

The solution to this technical problem is achieved by providing the embodiments characterised in the claims.

Accordingly, the present invention relates to an inhibitor of necroptosis for use in preventing the metastasis of tumours.

The term “inhibitor” as used herein refers to a compound that specifically reduces or abolishes the induction and/or execution of a signalling pathway, preferably by reducing or abolishing the biological function or activity of a component, more preferably a protein, which is required in the signalling pathway to be inhibited. An inhibitor may perform any one or more of the following effects in order to reduce or abolish the biological function or activity of the protein to be inhibited: (i)(a) the transcription of the gene encoding the protein to be inhibited is lowered, i.e. the level of mRNA is lowered, (i)(b) the transcription of a gene encoding a protein counteracting the protein to be inhibited is enhanced, (ii)(a) the translation of the mRNA encoding the protein to be inhibited is lowered, (ii)(b) the translation of the mRNA encoding a protein counteracting the protein to be inhibited is enhanced (iii)(a) the stability of the protein to be inhibited is lowered in the presence of the inhibitor (iii)(b) the stability of the protein counteracting the protein to be inhibited is enhanced in the presence of the inhibitor, (iv)(a) the protein to be inhibited performs its biochemical or cellular function with lowered efficiency in the presence of the inhibitor, (iv)(b) the protein counteracting the protein to be inhibited performs its biochemical or cellular function with enhanced efficiency in the presence of the inhibitor.

The term “protein” as used herein describes a group of molecules consisting more than 30 amino acids and is used interchangeably with the term “polypeptide” herein, whereas the term “peptide” as used herein describes a group of molecules consisting of up to 30 amino acids. The group of peptides and polypeptides are referred to together with the term “(poly)peptide”. Also encompassed by the term “(poly)peptide” are fragments of proteins of more than 30 amino acids. The term “fragment of protein” as used herein refers to a portion of a protein comprising at least the amino acid residues necessary to maintain the biological activity of the protein. Preferably, the amino acid chains are linear. (Poly)peptides may further form multimers consisting of at least two identical or different molecules. The corresponding higher order structures of such multimers are correspondingly termed homo- or heterodimers, homo- or heterotrimers etc. Furthermore, peptidomimetics of such (poly)peptides where amino acid(s) and/or peptide bond(s) have been replaced by functional analogues are also encompassed by the invention. Such functional analogues include all known amino acids other than the 20 gene-encoded amino acids, such as selenocysteine. The term “(poly)peptide” also refers to naturally modified (poly)peptides where the modification is effected e.g. by glycosylation, acetylation, phosphorylation and similar modifications which are well known in the art.

It is also well known that (poly)peptides are not always entirely linear. For instance, (poly)peptides may be branched as a result of ubiquitination, and they may be circular, with or without branching, generally as a result of post-translation events, including natural processing event and events brought about by human manipulation which do not occur naturally. Circular, branched and branched circular (poly)peptides may be synthesized by non-translational natural processes and by synthetic methods. The modifications can be a function of how the (poly)peptide is made. For recombinant (poly)peptides, for example, the modifications will be determined by the host cells posttranslational modification capacity and the modification signals in the amino acid sequence. Accordingly, when glycosylation is desired, a (poly)peptide should be expressed in a glycosylating host, generally an eukaryotic cell, for example Cos7, HELA or others. The same type of modification may be present in the same or varying degree at several sites in a given (poly)peptide. Also, a given (poly)peptide may contain more than one type of modification.

As used herein the term “a protein counteracting the protein to be inhibited” refers to a protein which either reduces or abolishes the expression (for example by acting as a transcriptional repressor), the stability (e.g. by targeting the protein to be inhibited for (proteasomal) degradation or by cleaving it into inactive fragments) or the activity (for example by directly binding to the protein to be inhibited thereby blocking its active site) of the protein to be inhibited or which mediates an effect that is opposed to that of the protein to be inhibited. For example, if the protein to be inhibited is a kinase the counteracting protein acting by the latter mechanism may be a phosphatase targeting the same substrate.

Compounds falling in class (i)(a) include compounds interfering with the transcriptional machinery and/or its interaction with the promoter of said gene and/or with expression control elements remote from the promoter such as enhancers. Compounds falling in class (i)(b) include compounds enhancing the activity/efficiency of the transcriptional machinery and/or its interaction with the promoter of said gene and/or with expression control elements remote from the promoter such as enhancers. Compounds of class (ii)(a) comprise antisense constructs and constructs for performing RNA interference (e.g. siRNA) well known in the art (see, e.g. Zamore (2001) Nat Struct Biol. 8(9), 746; Tuschl (2001) Chembiochem. 2(4), 239). Generally, the following holds true: Targeting on the nucleic acid level is often effected by hybridisation to the target sequence. The higher the level of identity of the targeting compound to the complementary sequence of the target nucleic acid, the higher the level of specificity of targeting. Class (ii)(b) encompasses compounds which enhance the translation of an mRNA for example by enhancing its stability. Compounds of class (iii)(a) lower the stability of the protein to be inhibited for example by interfering with its correct folding, by targeting it for (proteasomal) degradation and/or by inducing or mediating its cleavage into inactive fragments. Class (iii)(b) encompasses compounds which enhance the stability of a protein counteracting the protein to be inhibited for example by stabilising its correct folding and/or by preventing its (proteasomal) degradation or cleavage into inactive fragments. Compounds of class (iv)(a) interfere with the molecular or cellular function of the protein to be inhibited. As compounds inhibiting the molecular function, active site binding compounds (e.g. a small organic, an antibody, peptide or aptamer), in particular compounds capable of binding to the active site of protein to be inhibited, are envisaged. This also includes compounds which do not necessarily directly bind to the protein to be inhibited, but still interfere with the cellular activity of the protein, for example by binding to and/or inhibiting the function, or by inhibiting expression of members of the pathway in which the protein to be inhibited is involved. These members may be either upstream or downstream of the protein to be inhibited within the signalling pathway. Compounds of class (iv)(b) include those which enhance the molecular or cellular function of the protein counteracting the protein to be inhibited. This includes compounds which bind to the protein counteracting the protein to be inhibited, and thereby enhance the activity of the protein counteracting the protein to be inhibited, for example, by maintaining the active conformation of the counteracting protein. In this regard, a biochemical function designates a function of the protein per se, for example, the enzymatic function of a protein.

The cellular function of a protein comprises functions of the protein within the cellular context, e.g., a function which may only occur upon interaction with other cellular components, in particular other proteins.

The inhibitor, in accordance with the present invention, may in certain embodiments be provided as a proteinaceous compound or as a nucleic acid molecule encoding the inhibitor. For example, the nucleic acid molecule encoding the inhibitor may be incorporated into an expression vector comprising regulatory elements, such as for example specific promoters, and thus can be delivered into a cell. Methods for targeted transfection of cells and suitable vectors are known in the art, see for example Sambrook and Russel (“Molecular Cloning, A Laboratory Manual”, Cold Spring Harbor Laboratory, N.Y. (2001)). Incorporation of the nucleic acid molecule encoding the inhibitor into an expression vector allows to either selectively or permanently elevate the level of the encoded inhibitor in any cell or a subset of selected cells of a recipient. Thus, a tissue- and/or time-dependent expression of the inhibitor can be achieved, for example, restricted to endothelial cells. In a preferred embodiment, the inhibitor is therefore an endothelial cell-specific inhibitor. As used herein the term “endothelial cells” refers to thin, flattened cells, a layer of which makes up the endothelium which lines the inside surfaces of blood vessels and lymph vessels. Accordingly, the term “endothelial cells” encompasses vascular endothelial cells and lymphatic endothelial cells.

The term “necroptosis” is interchangeably used herein with the term “programmed necrosis” or “regulated necrosis”. Necroptosis is known in the art and is used herein accordingly to refer to a type of programmed cell death which resembles necrosis in its morphological features such as rounding of the cell, a gain in cell volume (also known as oncosis), organelle swelling, lack of internucleosomal DNA fragmentation, and plasma membrane rupture and which is dependent on the kinase activities of receptor-interacting protein kinase 1 (RIPK1) and/or RIPK3 (Krysko et al., 2008; Methods 44; 205-221). Thus, necroptosis is a type of programmed cell death that can be avoided, for example, by inhibiting the formation and/or necroptosis-promoting activity of the necrosome, and preferably the activity of RIPK1 and/or RIPK3, and/or the interaction of RIPK1 with RIPK3.

The term “necrosome” as used herein refers to an intracellular multiprotein complex comprising, as a core complex, RIPK1 and RIPK3. The necrosome complex further comprises the mixed lineage kinase domain-like protein (MLKL) (Sun et al. (2012), Cell 148:213-227). Tumour necrosis factor receptor type 1 associated death domain protein (TRADD) and FAS-associated protein with a death domain (FADD), caspase 8 and the serine/threonine-protein phosphatase phosphoglycerate mutase family member 5 (PGAMS) (Micheau et al., Cell 14:1814-190 (2003) and Wang et al. (2012), Cell 148:228-243) have also been described as components of the necrosome. If caspase 8 is activated, it cleaves RIPK1 and RIPK3 thereby preventing the effector mechanism of necroptosis (Vandenabeele et al., Nature Reviews Mol Cell Biol, 11:700-714 (2010)). Thus, caspase 8 is not required in the execution of necroptosis but instead acts as a negative regulator of this type of cell death. Within the necrosome RIPK1 and RIPK3 interact via their homotypic interaction motif (RHIM) domains. The kinase-like domain of MLKL binds to the kinase domain of RIPK3 thus recruiting MLKL into the necrosome (Dondelinger et al., 2014 Cell Reports 7, 071-981).

As used herein the term “RIPK1” refers to receptor interacting protein kinase 1 (also known as RIP1), a serine/threonine kinase comprising an N-terminal kinase domain, an intermediate domain (ID) comprising a RIP homotypic interaction motif (RHIM) and a C-terminal death domain (DD). Preferably, RIPK1 is characterised on the DNA level by SEQ ID NO:1 and on the amino acid level by SEQ ID NO:2. Via its DD RIPK1 is recruited, for example, to death receptors such as tumour necrosis factor receptor 1 (TNFR1), tumour necrosis factor related apoptosis inducing ligand inhibitor receptor 1 or 2 (TRAILRI or TRAILR2) or death receptor 6 (DR6) either via direct interaction or via DD-containing adaptor proteins such as tumour necrosis factor receptor type 1 associated death domain protein (TRADD) and FAS-associated protein with a death domain (FADD). Further, RIPK1 can also be recruited to toll-like receptors (TLR) and their respective adapter molecules. It is involved in prosurvival signalling pathways such as the activation of nuclear factor kappa B (NF-kB) as well as in the induction of cell death. The role of RIPK1 in prosurvival signalling is independent of its kinase activity. Similarly, also apoptosis may occur in the absence of RIPK1 kinase activity. However, specific forms of apoptosis as well as necroptosis depend on the kinase activity of RIPK1. The considerations with regard to inhibitors in general as provided herein above also apply to inhibitors of RIPK1 in particular. Thus, a RIPK1 inhibitor may for example decrease the expression of RIPK1 (e.g. an siRNA or shRNA specific for RIPK1), may decrease the stability of RIPK1 for example by increasing its modification with ubiquitin chains that target it for degradation or may interfere with its kinase activity. Preferably, the RIPK1 inhibitor is an inhibitor reducing or abolishing the kinase activity of RIPK1. Examples for inhibitors interfering with the kinase activity of RIPK1 are necrostatin-1 (5-(1H-indo1-3-ylmethyl)-3-methyl-2-thioxo-4-imidazolidinone, 5-(indo1-3-ylmethyl)-3-methyl-2-thio-hydantoin) and necrostatin-1 stable (5-((7-chloro-1 H-indol-3-yl)methyl)-3-methyl-2,4-imidazolidinedione, 5-((7-chloro-1H-indo1-3-yl)methyl)-3-methylimidazolidine-2,4-dione). Further, it has been described that also Nec-1 inactive (Nec1i; 5-((1H-indol-3-yl)methyl)-2-thioxoimidazolidin-4-one) which is approximately 100-times less effective than Nec-1 in vitro can efficiently inhibit necroptosis in cellular systems or in vivo where Nec1i can be modified to an active form (Takahashi et al., 2012, Cell Death Dis., 3, e437). Accordingly, depending on the application, also Nec1i can be used as a RIPK1 inhibitor.

As used herein the term “RIPK3” refers to a serine/threonine kinase of the RIP family which comprises a kinase domain and a C-terminal domain unique from other RIP family members which comprises a RHIM. Preferably, RIPK3 is characterised on the DNA level by SEQ ID NO:3 and on the amino acid level by SEQ ID NO:4. Upon induction of necrosis, RIPK3 interacts with, and phosphorylates RIPK1 and MLKL to form a necrosis-inducing complex. The interaction with RIPK1 is mediated via the RHIM domains of the kinases whereas the kinase-like domain of MLKL binds to the kinase domain of RIPK3. RIPK1 and RIPK3 undergo reciprocal auto- and trans-phosphorylation. Phosphorylation of Ser-199 plays a role in the necroptotic function of RIPK3. Further, RIPK3 has also been described to phosphorylate PGAM5. The general considerations with regard to inhibitors also apply for inhibitors specific for RIPK3. Thus an inhibitor may, for example, affect the expression, the stability and/or the kinase activity of RIPK3. Inhibitors affecting the expression of RIPK3 may be an siRNA or an shRNA specific for RIPK3. Further exemplary specific RIPK3 inhibitors are GSK840, GSK843 and KSK872 as described in Mandal et al. Mol Cell 56, 481-495 (2014) and Silke et al., Nat Imm 16, 689-697 (2015).

The term “MLKL” refers to mixed lineage kinase domain-like protein, a pseudokinase reported to be involved in necroptosis induction. In humans two isoforms of this protein are known. Isoform 1 is characterised on the DNA level by SEQ ID NO:5 and on the amino acid level by SEQ ID NO:6. SEQ ID NO:7 represents isoform 2 on the DNA level and SEQ ID NO:8 shows the amino acid sequence of this isoform. Preferably, MLKL is characterised on the DNA level by SEQ ID NO:5 and on the amino acid level by SEQ ID NO:6. Structurally, MLKL comprises an N-terminal four-helical bundle tethered to the C-terminal pseudokinase domain, which contains an unusual pseudoactive site. Although the pseudokinase domain binds ATP, it is catalytically inactive. Phosphorylation by RIPK3 at T357 and 5358 induces a conformational switch in MLKL. This conformational change induces homotrimerisation or oligornerisation of MLKL and translocation of the trimer or oligomer to intracellular and plasma membranes. It has been described that at the membranes the trimer or oligomer either induces calcium or sodium influx or directly permeabilises the membrane. As an alternative mechanism it has been suggested that MLKL is involved in recruiting PGAM5 to the necrosome. Overall, MLKL's essentially nonenzymatic role in necroptotic signalling is induced by receptor-interacting serine-threonine kinase 3 (RIPK3)-mediated phosphorylation. Further it has been reported that MLKL is inhibited by necrosulfonamide ((E)-N-(4-(N-(3-methoxypyrazin-2-yl)su lfamoyl)phenyl)-3-(5-nitrothiophene-2-yl)acrylamide), a specific inhibitor of necroptosis that targets Cys-86 of MLKL. Alternatively, an inhibitor of MLKL may be of any of the general inhibitor classes described herein above. For example, the inhibitor may be an siRNA or shRNA specific for MLKL.

Necroptosis can be induced by a variety of stimuli, including, for example, members of the tumour necrosis factor receptor (TNFR) family. Induction of necroptosis often requires apoptosis to be inhibited, for example, by caspases being absent or inactivated. Once necroptosis is initiated, the necrosome is formed. It was reported that the necroptotic signal is further transduced by binding and activation of the phosphatase PGAM5 and dynamin-related protein 1 (DRP1)-mediated mitochondrial fragmentation. In addition, depletion of ATP and reactive oxygen species (ROS) have been implicated as critical mediators of necroptotic cell death. As an alternative mechanism for the execution of necroptosis it has been suggested that MLKL translocates to the plasma membrane, multimerises and either triggers extracellular calcium influx from the transient receptor potential melastatin-related 7 (TRPM7) (Cai et al., 2014, Nat. Cell Biol. 16(1):55-65), causes sodium influx (Chen et al., 2014; Cell. Res. 24(1):105-21) or directly permabilises the plasma membrane potentially by forming a pore (Dondelinger et a., 2014, Cell Reports 7, 971-981; Wang et al., Mol. Cell. 54(1):133-46). These events characterise necroptosis and ultimately lead to the necroptotic death of the cell. Contrary to necroptosis which is a programmed form of cell death and can be avoided by inhibiting, for example, the formation of the necrosome and/or the necroptosis-inducing activity of the necrosome, necrosis is an accidental form of cell death. The possibility of inhibiting necroptosis represents convenient means to discriminate between necroptosis (programmed necrosis) and accidental cell death in the form of necrosis (Kromer et al., 2009, Cell Death Diff. 16(1): 3-11).

As used herein the term “necroptosis inhibitor” refers to a compound that specifically reduces or abolishes the induction and/or execution of the signalling pathway resulting in necroptotic cell death, preferably by reducing or abolishing the biological function or activity a component, more preferably a protein, which is involved in this signalling pathway. Non-protein components of the necroptosis pathway which can also be targeted by a necroptosis inhibitor include, for example, reactive oxygen species (ROS) and calcium ions (Ca²⁺). Compounds regulating intracellular levels of ROS (ROS-inhibitors) include, for example, antioxidants such as ascorbate and N-acetyl-L-cysteine (NAC). The necroptotic effect of Ca²⁺ can be inhibited, for example by chelating agents or calcium channel blockers.

Preferably, the necroptosis inhibitor is not a ROS inhibitor. In general, a necroptosis inhibitor prevents a cell from undergoing necroptosis. Preferably, a necroptosis inhibitor specifically inhibits a protein which is required in the necroptosis pathway. More preferably, a necroptosis inhibitor specifically inhibits the formation of the necrosome and/or the necroptosis-promoting activity of the necrosome.

The term “specifically” in this context refers to the capability of an inhibitor to not have an effect or an essential effect on other molecules than the target molecules. In other words, a corresponding inhibitor does not display cross-reactivity or essentially does not display cross-reactivity. In this context, the term “essentially” is meant to refer to an insignificant or negligible effect. The insignificance or negligibility can be based on functional or quantitative parameters. For example, only a minimal amount of cross-reactivity occurs with a different non-target molecule. Preferably, the expression and/or activity of a non-target molecule is inhibited by not more than 20%, such as not more than 15%, preferably by not more than 10%, such as not more than 5%, such as not more than 2%, more preferably by not more than 1% as compared to the expression and/or activity in the absence of the inhibitor. Further it is preferred that the cross-reactivity with a different non-target molecule is associated only with a negligible or insignificant effect, if any. In this regard it is preferred that the biological or cellular function effected or contributed to by the non-target molecule is reduced by less than 20%, such as less than 15%, preferably by less than 10%, such less than 5%, such as less than 2%, more preferably by less than 1% as compared to the biological or cellular function in the absence of the inhibitor. Most preferably, the cross-reactivity with a different non-target molecule is not associated with any effect, i.e. the biological or cellular function effected or contributed to by the non-target molecule is not reduced. In particular, a necroptosis inhibitor specifically inhibiting necroptosis does not exhibit cross-reactivity with a compound important for the execution of apoptosis. In other words, a necroptosis inhibitor in accordance with the present invention does not inhibit apoptosis, i.e. the inhibitor is not an apoptosis inhibitor.

The term “necroptosis inhibitor” thus encompasses compounds that target components (1) on a cell or secreted or released by a cell that induce necroptosis in other cells, such as e.g. a ligand which is expressed by one cell and induces necroptosis in another cell (the target cell) upon binding to a receptor expressed on said target cell, (2) of the signalling pathway upstream of the necrosome in an endothelial cell, (3) that target the necrosome itself, preferably targeting RIPK1 and/or RIPK3 activity, and/or the interaction of RIPK1 and RIPK3 in an endothelial cell, and (4) that prevent the signal progression and execution of necroptosis downstream of the necrosome in an endothelial cell. With regard to the compounds in class (1) to (4), compounds of class (2) and (3) are preferred and even more preferred are compounds of class (3).

The above types of compounds inhibiting necroptosis can belong to one of the inhibitor classes (i) to (iv) described herein above: (i)(a) the transcription of a gene encoding a protein effecting or contributing to effecting necroptosis (also referred to herein as pro-necroptotic protein) is lowered or abolished (e.g. the genes encoding RIPK1, RIPK3 or MLKL, signalling components required for activating the necrosome upstream of the necrosome (for instance signalling components like the death receptor such as, e.g., DR6, a corresponding ligand which is expressed on or secreted by the metastasising tumour cell such as, for example amyloid beta precursor protein (APP) which was shown to bind to DR6 and to induce cell death (Nikolaev et al., 2009, Nature, 457(7232):981-9) and which is expressed by several tumour cells (Woods & Padmanabhan, 2013, JBC, 288(42):30114-24), adapter proteins such as TRADD, FADD, toll-interleukin-1 receptor domain-containing adapter protein inducing interferon beta (TRIF), tumour necrosis factor receptor-associated factor (TRAF) and myeloid differentiation primary response protein (Myd88) or deubiqutinases such as CYLD or A20) or cellular components required for execution of necroptosis downstream of the necrosome); (i)(b) the transcription of a gene encoding a protein preventing necroptosis (also referred to herein as an anti-necroptotic protein) is enhanced (e.g. the gene encoding caspase 8, clAP1, clAP2, or TAK1); (ii)(a) the translation of a gene encoding a pro-necroptotic protein is lowered or abolished (e.g. the genes encoding RIPK1, RIPK3 or MLKL, signalling components required for activating the necrosome upstream of the necrosome (for instance signalling components like the death receptor such as, e.g., DR6, the corresponding ligand which is expressed on or secreted by the metastasising tumour cell, such as, for example APP, adapter proteins such as TRADD, FADD, toll-interleukin-1 receptor domain-containing adapter protein inducing interferon beta (TRIF), tumour necrosis factor receptor-associated factor (TRAF) and myeloid differentiation primary response protein (Myd88) or deubiqutinases such as CYLD or A20), or cellular components required for execution of necroptosis downstream of the necrosome); (ii)(b) the translation of a gene encoding an anti-necroptotic protein is enhanced (e.g. the gene encoding caspase 8, clAP1, clAP2, or TAK1); (iii)(a) the stability of a protein effecting or contributing to effecting necroptosis is lowered or abolished (e.g. RIPK1, RIPK3 or MLKL, signalling components required for activating the necrosome upstream of the necrosome (for instance signalling components like the death receptor such as, e.g., DR6, the corresponding ligand which is expressed on or secreted by the metastasising tumour cell, such as, for example APP, adapter proteins such as TRADD, FADD, toll-interleukin-1 receptor domain-containing adapter protein inducing interferon beta (TRIF), tumour necrosis factor receptor-associated factor (TRAF) and myeloid differentiation primary response protein (Myd88) or deubiqutinases such as CYLD or A20) or cellular components required for execution of necroptosis downstream of the necrosome); (iii)(b) the stability of a protein preventing necroptosis is enhanced (e.g. caspase 8, clAP1, clAP2, or TAK1), (iv)(a) a pro-necroptotic protein (e.g. RIPK1, RIPK3, MLKL, CYLD or A20) performs its biochemical or cellular function with lowered efficiency or has abolished biochemical or cellular function in the presence of the necroptosis inhibitor; (iv)(b) an anti-necroptotic protein (e.g. caspase 8, cIAP1, cIAP2, or TAK1) performs its biochemical or cellular function with enhanced efficiency or has enhanced biochemical or cellular function in the presence of the necroptosis inhibitor.

In this regard, the term “lowered” means that the transcription of a gene or efficiency of a protein is lowered with increasing preference at least 2-fold, at least 5-fold, at least 10-fold, at least 50-fold and at least 100-fold as compared to the transcription or efficiency in the absence of the necroptosis inhibitor. The term “enhanced” means that the transcription of a gene or efficiency of a protein is enhanced with increasing preference at least 2-fold, at least 5-fold, at least 10-fold, at least 50-fold and at least 100-fold as compared to the transcription or efficiency in the absence of the necroptosis inhibitor.

As described above, compounds of class (ii)(a) comprise antisense constructs and constructs for performing RNA interference (e.g. siRNA) well known in the art (see, e.g. Zamore (2001) Nat Struct Biol. 8(9), 746; Tuschl (2001) Chembiochem. 2(4), 239). In particular antisense constructs and constructs for performing RNA interference which specifically target RIPK1, RIPK3 or MLKL, preferably RIPK3 or MLKL, are envisaged. It is known in the art how such constructs are designed. Exemplary siRNAs for RIPK1, RIPK3 and MKLK, respectively, have been described and are commercially available, for example, from SIGMA or Qiagen. Class (ii)(b) encompasses compounds stabilising the mRNA of gene encoding an anti-necroptotic protein (e.g. the gene encoding caspase 8, cIAP1, cIAP2, or TAK1) or enhancing its translation in another manner. The translation of (m)RNA to protein and the stability of the protein itself can be regulated by specific non-coding (nc) RNA. These include micro-RNA, long-non-coding-RNA, circular-RNA and others. Usually, the activity of ncRNA results in a degradation of the respective mRNA and/or protein and thus lowers the amount of the protein. Therefore, an inhibitor of this class may indirectly increase the translation and, thus, the level of an anti-necroptotic protein by blocking a corresponding ncRNA. Class (iii)(a) includes compounds which lower the stability of a pro-necroptotic protein for example by interfering with its correct folding and/or by targeting it for proteasomal degradation. This may include ubiquitin ligases specifically targeting the pro-necroptotic protein for proteasomal degradation or proteases specifically cleaving the pro-necroptotic protein into inactive fragments. Compounds falling within class (iii)(b) enhance the stability of an anti-necroptotic protein. Thus, this class includes, for example, compounds inhibiting ubiquitin ligases or proteases that, in absence of the compound, lead to the degradation or cleavage of the anti-necroptotic protein. Also encompassed are compounds directly binding to the anti-necroptotic protein and stabilising its correct folding. Compounds of class (iv)(a) interfere with the molecular or cellular function of a pro-necroptotic protein, in particular with the induction and/or execution of necroptosis. Accordingly, active site binding compounds (e.g. a small organic, an antibody, peptide or aptamer), in particular compounds capable of binding to the active site of the kinases RIPK1 or RIPK3, are envisaged. More preferred are compounds specifically binding to an active site of RIPK3. Also envisaged are compounds which bind to or block the binding sites of a pro-necroptotic protein thereby blocking these sites for other interaction partners. The latter group of compounds which block binding sites of the protein may be fragments or modified fragments with improved pharmacological properties of the naturally occurring binding partners. In this regard, it is preferred that the compound blocks the binding of RIPK1 with RIPK3, the dimerisation of RIPK1 or RIPK3 or the interaction between MLKL and RIPK3.

For example, it has been reported that proteins or peptides containing a RHIM sequence (consensus sequence IN-Q-1/UV-G-x-x-N-x-M/L/I) may disrupt the interaction between RIPK1 and RIPK3 and may thus inhibit necroptosis (Mack et al., 2008, PNAS 105, 3094-3099; Kaiser et al., 2008, J. Immunol. 181, 6427-6434). Also dominant-negative versions of pro-necroptotic proteins are envisaged. Such dominant-negative proteins maintain the ability to bind to interaction partners and thus compete with their naturally occurring active counterpart. At the same time they lack the activity of the naturally occurring variant. For example, a kinase-dead variant of RIPK1 or RIPK3 can still be recruited to the necrosome, thus competing with the naturally occurring active protein, but cannot phosphorylate the targets of RIPK1 or RIPK3 thus blocking necroptosis. Class (iv)(b) includes compounds which enhance the molecular or cellular function of an anti-necroptotic proteins. This may encompass a constitutively active form of the anti-necroptotic protein, such as, for example, a constitutively active form of TAK1.

The efficiency of “a necroptosis inhibitor” can be determined by comparison to a negative control and/or a positive control as defined and further detailed herein. As used herein a “positive control” designates compounds which in conditions/experimental setups for testing necroptosis show results which are already known or expected to be positive, i.e. a positive control will result in the inhibition of necroptotic cell death after a predetermined time and may be a known necroptosis inhibitor. An example of a positive control is necrostatin-1. As used herein a “negative control” designates compounds which in conditions/experimental setups for testing necroptosis show results which are already known or expected to be negative, i.e. a negative control will result in the occurrence of necroptotic cell death after a predetermined time and may be an inactive compound, e.g. an inactive necrostatin analogue.

As mentioned herein above, a necroptosis inhibitor prevents a cell from undergoing necroptosis. In this regard, the term “prevent” includes also reducing necroptotic cell death which means that the necroptotic cell death is reduced by the necroptosis inhibitor with increasing preference at least by 20%, at least by 30%, at least by 40%, at least by 50%, at least by 75%, at least by 90% and at least by 95% as compared to the negative control after a predetermined time.

As used herein the term “a predetermined time” specifies a period of time which is sufficient for the induction of necroptotic cell death. It is known in the art how to determine such a time period. With increasing preference a predetermined time is at least 6 h, at least 12 h, at least 24 h, at least 48 h or at least 72 h.

It is of note that whereas the controls are preferably included into the experimental setup, this is by no means necessary. A control may also be, for example, a statistical readout of cell death obtained from control experiments using the above types of compounds previously carried out. A statistical readout is in general obtained by averaging more than one control experiments previously carried out. The average may be obtained by weighting the results from repeating the same control experiment or by weighting the results from different control experiments. Statistical methods for obtaining a statistical readout are well-known in the art. The statistical readout is then compared to results obtained testing a compound for its ability to act as a necroptosis inhibitor.

In a preferred embodiment, the inhibitor reduces necroptosis by at least 50%, preferably by at least 75%, more preferred by at least 90% and even more preferred by at least 95% such as at least 98% or even by 100% as compared to the necroptosis in the absence of said inhibitor. Depending on said mechanism of action, a reduction of, e.g., 75% may be achievable by a given inhibitor by reducing the formation and/or necroptosis-inducing activity of all or substantially all of the necrosomes by 75% or by fully inhibiting 75% of all or substantially all of the necrosomes. In other words, the reduction of said biological function or activity may be of qualitative or quantitative nature. The term “substantially all” is meant to specify that at least 95% or more of the necrosomes are encompassed. The use of the term “substantially all” is a tribute to the constant changes of protein expression and resulting signalling events observed in a cell that may prevent truly addressing all of the necrosomes. Preferably, all necrosomes are fully inhibited.

Necroptotic cell death can be quantified for example by analysing the morphological features by electron microscopy or by indirect methods comprising the quantification of cell death in the presence and absence of pharmacological or genetic inhibition of one of the types of cell death, e.g. in the presence of apoptosis or necroptosis inhibitors. Preferably, the method described in the Examples herein below is used in order to determine quantity and quality of cell death. In this regard, “determining the quality of cell death” refers to analysing by which form of cell death (e.g., apoptosis, necroptosis, necrosis) a cell has died or is dying. In other words, it is preferred that the method described in the Examples herein below is employed in order to determine the number of endothelial cells which have died or are dying by necroptosis.

The function of any of the inhibitors referred to in the present invention may be identified and/or verified by using, e.g., high throughput screening assays (HTS). High-throughput assays, independently of being biochemical, cellular or other assays, generally may be performed in wells of microtiter plates, wherein each plate may contain, for example 96, 384 or 1536 wells. Handling of the plates, including incubation at temperatures other than ambient temperature, and bringing into contact of test compounds with the assay mixture is preferably effected by one or more computer-controlled robotic systems including pipetting devices. In case large libraries of test compounds are to be screened and/or screening is to be effected within short time, mixtures of, for example 10, 20, 30, 40, 50 or 100 test compounds may be added to each well. In case a well exhibits biological activity, said mixture of test compounds may be de-convoluted to identify the one or more test compounds in said mixture giving rise to the observed biological activity.

The determination of the binding of potential inhibitors can be effected in, for example and without limitation, any binding assay, such as a biophysical binding assay, which may be used to identify binding of test molecules prior to performing the functional/activity assay with the inhibitor. Suitable biophysical binding assays are known in the art and comprise fluorescence polarisation (FP) assay, fluorescence resonance energy transfer (FRET) assay and surface plasmon resonance (SPR) assay. Instead of or in addition to assessing the direct interaction of inhibitor and target molecule by binding assays, one may indirectly determine the interaction of the inhibitor with its target molecule by using a suitable read out. For example, in cases where the inhibitor acts by decreasing the expression level of the target protein, the determination of the expression level of the protein can, for example, be carried out on the nucleic acid level or on the amino acid level.

Methods for determining the expression of a protein on the nucleic acid level include, but are not limited to, northern blotting, PCR, RT-PCR or real RT-PCR.

A northern blot allows the determination of RNA or isolated mRNA in a sample. Northern blotting involves the use of electrophoresis to separate RNA samples by size and detection with a hybridisation probe complementary to part of or the entire target sequence. Initially, typically total RNA extraction from the sample is performed. If desired, mRNA can be separated from said initial RNA sample through the use of oligo (dT) cellulose chromatography to isolate only the RNA with a poly(A) tail. RNA samples are then separated by gel electrophoresis. The separated RNA is then transferred to a nylon membrane through a capillary or vacuum blotting system. After transfer to the membrane, the RNA is immobilised through covalent linkage to the membrane by, e.g., UV light or heat. Then, the RNA is detected using suitably labelled probes and X-ray film and can subsequently be quantified by densitometry. Suitable compositions of gels, buffers and labels are well-known in the art and may vary depending on the specific sample and target to be identified. The above protocol is typical but by no means limiting for a northern blot.

PCR is well known in the art and is employed to make large numbers of copies of a target sequence. This is done on an automated cycler device, which can heat and cool containers with the reaction mixture in a very short time. The PCR, generally, consists of many repetitions of a cycle which consists of: (a) a denaturing step, which melts both strands of a DNA molecule and terminates all previous enzymatic reactions; (b) an annealing step, which is aimed at allowing the primers to anneal specifically to the melted strands of the DNA molecule; and (c) an extension step, which elongates the annealed primers by using the information provided by the template strand. Generally, PCR can be performed, for example, in a 50 pl reaction mixture containing 5 μl of 10× PCR buffer with 1.5 mM MgCl₂, 200 μM of each deoxynucleoside triphosphate, 0.5 μl of each primer (10 μM), about 10 to 100 ng of template DNA and 1 to 2.5 units of Taq polymerase. The primers for the amplification may be labelled or be unlabelled. DNA amplification can be performed, e.g., with a Applied Biosystems Veriti® Thermal Cycler (Life Technologies Corporation, Carlsbad, Calif.), C1000™ thermal cycler (Bio-Rad Laboratories, Hercules, Calif.), or SureCycler 8800 (Agilent Technologies, Santa Clara, Calif.): 2 min at 94° C., followed by 30 to 40 cycles consisting of annealing (e. g. 30 s at 50° C.), extension (e. g. 1 min at 72° C., depending on the length of DNA template and the enzyme used), denaturing (e. g. 10 s at 94° C.) and a final annealing step, e.g. at 55° C. for 1 min as well as a final extension step, e.g. at 72° C. for 5 min. Suitable polymerases for use with a DNA template include, for example, E. coli DNA polymerase I or its Klenow fragment, T4 DNA polymerase, Tth polymerase, Taq polymerase, a heat-stable DNA polymerase isolated from Thermus aquaticus Vent, Amplitaq, Pfu and KOD, some of which may exhibit proof-reading function and/or different temperature optima. However, it is well known in the art how to optimise

PCR conditions for the amplification of specific nucleic acid molecules with primers of different length and/or composition or to scale down or increase the volume of the reaction mix. The “reverse transcriptase polymerase chain reaction” (RT-PCR) is used when the nucleic acid to be amplified consists of RNA. The term “reverse transcriptase” refers to an enzyme that catalyzes the polymerisation of deoxyribonucleoside triphosphates to form primer extension products that are complementary to a ribonucleic acid template. The enzyme initiates synthesis at the 3′-end of the primer and proceeds toward the 5′-end of the template until synthesis terminates. Examples of suitable polymerising agents that convert the RNA target sequence into a complementary, copy-DNA (cDNA) sequence are avian myeloblastosis virus reverse transcriptase and Thermus thermophilus DNA polymerase, a thermostable DNA polymerase with reverse transcriptase activity marketed by Perkin Elmer. Typically, the genomic RNA/cDNA duplex template is heat denatured during the first denaturation step after the initial reverse transcription step leaving the DNA strand available as an amplification template. High-temperature RT provides greater primer specificity and improved efficiency. U.S. patent application Ser. No. 07/746,121, filed Aug. 15, 1991, describes a “homogeneous RT-PCR” in which the same primers and polymerase suffice for both the reverse transcription and the PCR amplification steps, and the reaction conditions are optimised so that both reactions occur without a change of reagents. Thermus thermophilus DNA polymerase, a thermostable DNA polymerase that can function as a reverse transcriptase, can be used for all primer extension steps, regardless of template. Both processes can be done without having to open the tube to change or add reagents; only the temperature profile is adjusted between the first cycle (RNA template) and the rest of the amplification cycles (DNA template). The RT reaction can be performed, for example, in a 20 μl reaction mix containing: 0.5 to 1 μg total RNA, approximately 10 to 30 pmol of the forward and reverse primer, respectively, 2 μl of 10× RT-PCR buffer, 0.2 μl AMV reverse transcriptase (25 U/μl ), 1 μl Taq Polymerase (5 U/μl ), a ribonuclease inhibitor and H₂O up to 20 μfinal volume.

The reaction may be, for example, performed by using the following conditions: The reaction is held at 45° C. for 30 min to allow for reverse transcription. The reaction temperature is then raised to 94° C. for 5 minutes to stop the reverse transcriptase reaction and to denature the RNA-cDNA duplex. Next, the reaction temperature undergoes 30 to 40 cycles of 94° C. for 30 seconds, 60° C. for 45 seconds and 72° C. for 1-3 minutes. Finally, the reaction temperature is held at 72° C. for 7 minutes for the final extension step, cooled to 15°, and held at that temperature until further processing of the amplified sample. Any of the above mentioned reaction conditions may be scaled up according to the needs of the particular case. The resulting products, e.g., are loaded onto an agarose gel and band intensities are compared after staining the nucleic acid molecules with an intercalating dye such as ethidium bromide or SybrGreen. A lower band intensity of the sample treated with the inhibitor as compared to a non-treated sample indicates that the inhibitor successfully inhibits the protein.

Real-time PCR employs a specific probe, in the art also referred to as TaqMan probe, which has a reporter dye covalently attached at the 5′ end and a quencher at the 3′ end. After the TaqMan probe has been hybridised in the annealing step of the PCR reaction to the complementary site of the polynucleotide being amplified, the 5′ fluorophore is cleaved by the 5′ nuclease activity of Taq polymerase in the extension phase of the PCR reaction. This enhances the fluorescence of the 5′ donor, which was formerly quenched due to the close proximity to the 3′ acceptor in the TaqMan probe sequence. Thereby, the process of amplification can be monitored directly and in real time, which permits a significantly more precise determination of expression levels than conventional end-point PCR. Also of use in real time RT-PCR experiments is a DNA intercalating dye such as SybrGreen for monitoring the de novo synthesis of double stranded DNA molecules.

Methods for the determination of the expression of a protein on the amino acid level include, but are not limited to, western blotting or polyacrylamide gel electrophoresis in conjunction with protein staining techniques such as Coomassie Brilliant blue or silver-staining. The total protein is loaded onto a polyacrylamide gel and electrophoresed. Afterwards, the separated proteins are transferred onto a membrane, e.g. a polyvinyldifluoride (PVDF) membrane, by applying an electrical current. The proteins on the membrane are exposed to an antibody specifically recognising the protein of interest. After washing, a second antibody specifically recognising the first antibody and carrying a readout system such as a fluorescent dye is applied. The amount of the protein of interest is determined by comparing the fluorescence intensity of the protein derived from the sample treated with the inhibitor and the protein derived from a non-treated sample. A lower fluorescence intensity of the protein derived from the sample treated with the inhibitor indicates a successful inhibitor of the protein. Also of use in protein quantification is the Agilent Bioanalyzer technique (e.g., Agilent 2100 Bioanalyzer; Agilent Technologies, Santa Clara, Calif.).

Preferably, the inhibitor of the present invention is comprised in a pharmaceutical composition, preferably further comprising a pharmaceutically acceptable carrier, excipient and/or diluent.

The term “pharmaceutical composition”, as used herein, relates to a composition for administration to a patient, preferably a human patient. The pharmaceutical composition of the invention comprises at least one, such as at least two, e.g. at least three, in further embodiments at least four such as at last five of the inhibitors mentioned herein. In cases where more than one inhibitor is comprised in the pharmaceutical composition it is understood that none of these inhibitors has any or any essentially inhibitory effect on the other inhibitors also comprised in the composition. The term “essentially” in this context refers to an insignificant or negligible inhibitory effect. Again, it is preferred that the inhibitors provide an additive effect and, optionally, a synergistic effect in their inhibitory activity.

The composition may be in solid, liquid or gaseous form and may be, inter alia, in a form of (a) powder(s), (a) tablet(s), (a) solution(s) or (an) aerosol(s).

As mentioned above, it is preferred that said pharmaceutical composition comprises a pharmaceutically acceptable carrier, excipient and/or diluent. Examples of suitable pharmaceutical carriers, excipients and/or diluents are well known in the art and include phosphate buffered saline solutions, water, emulsions, such as oil/water emulsions, various types of wetting agents, sterile solutions etc. The carriers, excipients and diluents to some extent depend on the chemical nature of the actual inhibitors and can be chosen according to established protocols. Compositions comprising such carriers can be formulated by well known conventional methods. These pharmaceutical compositions can be administered to the subject at a suitable dose. Administration of the suitable compositions may be effected by different ways, e.g., by intravenous, oral, rectal, intraperitoneal, subcutaneous, intramuscular, topical, intradermal, intranasal or intrabronchial administration. It is preferred that the mode of administration is a systemic administration such as, e.g., carried out by injection and/or delivery, e.g., to a site in the bloodstream such as a coronary artery. The compositions may also be administered directly to the target site, e.g., by biolistic delivery to an external or internal target site, preferably the site where the tumour is located. In cases where the region of metastasis or the organ to which tumour cells metastasise is small and defined, localised administration may be preferred over systemic administration. In these cases, local administration may be advantageous to, e.g., minimise the amount of drug used or decrease the risk of adverse side effects, if any. A combination of systemic and localised administration may be chosen in order to achieve a temporally increased concentration of the inhibitor as defined herein at a specific site in the body. The dosage regimen will be determined by the attending physician and clinical factors. As is well known in the medical arts, dosages for any one patient depend upon many factors, including the patients size, body surface area, age, the potency and bioavailability of the particular compound to be administered, sex, time and route of administration, general health, and other drugs being administered concurrently. In particular, the potency and mode of action of an inhibitor may dictate not only its dosage, but also its way of administration. Pharmaceutically active matter may be present in amounts between 1 ng and 10 mg/kg body weight per dose; however, doses below or above this exemplary range are envisioned, especially considering the aforementioned factors. If the regimen is a continuous infusion, it should also be in the range of 0.01 pg to 10 mg units per kilogram of body weight per minute. The continuous infusion regimen may be completed with a loading dose in the dose range of 1 ng and 10 mg/kg body weight.

In the case of localised delivery of the inhibitors of the present invention to the endothelium or to the tumour(s), respectively, various options exist to achieve said site-specific delivery to the endothelium or the tumour(s). For example, the inhibitor(s) may be functionalised in that moieties are attached that specifically target the endothelium or tumour cells, respectively. Based on the specific expression of defined antigens by endothelial cell and tumour cells, respectively, inhibitors may be attached to antibodies or antibody fragments specifically interacting with the antigen expressed by the respective cell, i.e. by the endothelial cell or the tumour cell. Alternatively, the vehicle carrying the inhibitor(s) may be functionalised so that specifically tumour cells or endothelial cells are targeted. In cases where tumour cells exhibit particular enzymatic activities or uptake mechanisms, inhibitors can be functionalised in a way that it is converted by the enzymatic activity into a functionally active form or that it is subject to the specific uptake mechanism, respectively. Localised delivery to the tumour(s) is envisaged in particular for necroptosis inhibitors of class (1), i.e. for inhibitors that target components which are expressed on or secreted or released by a tumour cell and which induce necroptosis in endothelial cells.

Progress can be monitored by periodic assessment. The compositions may be administered locally or systemically. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like.

Preservatives and other additives may also be present such as, for example, antimicrobials, anti-oxidants, chelating agents, and inert gases and the like. It is particularly preferred that said pharmaceutical composition comprises further agents known in the art to antagonise heart failure. Since the pharmaceutical preparation relies on the above mentioned inhibitors, it is preferred that those mentioned further agents are only used as a supplement, i.e. at a reduced dose as compared to the recommended dose when used as the only drug, so as to e.g. reduce side effects conferred by the further agents. Conventional excipients include binding agents, fillers, lubricants and wetting agents.

The term “tumour” as used herein relates to neoplasms, e.g. characterised by abnormal tissue proliferation, that are known to or are suspected to metastasise and thus warrant the use of inhibitors as defined herein to prevent metastasis. Tumours that are known to metastasise are malignant tumours (also referred to in the art as cancerous tumours) in contrast to benign tumours that lack the ability to form metastases. However, many types of the latter tumours have the potential to become malignant, i.e. metastasise, (such as, e.g., teratomas). Where metastasis is to be expected the use of the inhibitors as defined herein to prevent metastasis may be warranted. Tumours in accordance with the invention are i) carcinoma, ii) sarcoma, iii) tumours of the blood-forming tissues, iv) germ cell tumours, v) blastoma, vi) mixed type tumours. For example, a tumour according to i) occurs in epithelial tissues, which cover the outer body and line mucous membranes and the inner cavitary structures of organs e.g. such as the breast, lung, the respiratory and gastrointestinal tracts, the endocrine glands, and the genitourinary system. Ductal or glandular elements may persist in epithelial tumours, as in adenocarcinomas like, e.g., thyroid adenocarcinoma, gastric adenocarcinoma, uterine adenocarcinoma. Tumours of the pavement-cell epithelium of the skin and of certain mucous membranes, such as e. g. tumours of the tongue, lip, larynx, urinary bladder, uterine cervix, or penis, may be termed epidermoid or squamous-cell carcinomas of the respective tissues and are in the scope of the definition of tumour as well. A tumour according to ii) develops in connective tissues, including fibrous tissues, adipose (fat) tissues, muscle, blood vessels, bone, and cartilage like e.g. osteogenic sarcoma, liposarcoma, fibrosarcoma, or synovial sarcoma. A tumour according to iii) may be a cancer of the blood (leukemia) or the lymphatic system (lymphoma). Leukaemias are a group of cancers of the blood-forming cells. They usually start in the bone marrow and the abnormal cells spread from there into the bloodstream and to other parts of the body. The leukaemia is described as lymphoid or myeloid, depending on which type of blood-forming cell in the bone marrow the abnormal leukaemia cells develop from. Further, leukemias are categorised according to whether they are acute or chronic and also according to which type of blood-forming cell has become cancerous. Accordingly, there are four main types of leukaemia: acute lymphoblastic leukaemia (ALL), acute myeloid leukaemia (AML), chronic lymphocytic leukaemia (CLL) and chronic myeloid leukaemia (CML). Lymphoma are derived from lymphocytes and can be subdivided in the two main categories of Hodgkin lymphoma (HL) (characterised by the presence of Reed-Sternberg cells) and non-Hodgkin lymphoma (NHL). The latter are further subdivided into several subcategories. A tumour according to iv) is a tumour derived from germ cells. It may occur in ovary or testes but also outside these organs such as e.g. head, chest, abdomen, pelvis, and sacrococcygeal. These tumour include germinomas, endodermal sinus tumour or yolk sac tumours, choriocarcinoma and embryonal carcinoma. Germinomas are also termed dysgerminoma when located in the ovaries; and seminoma when located in the testes. Choriocarcinoma is a very rare, germ cell tumour that arises from the cells in the chorion layer of the placenta. A tumour according to v) originates from precursor cells or blasts (immature or embryonic tissue) such as, for example, hepatoblastoma, medulloblastoma, nephroblastoma, neuroblastoma, pancreatoblastoma, pleuropulmonary blastoma, retinoblastoma and glioblastoma multiforme. A tumour according to vi) comprises tumour cells of different types. The different types may belong to the same or to different of the above categories. An example is carcinosarcoma. A carcinosarcoma is a tumour that develops in both epithelial and connective tissue. They may arise due to a carcinogenic event simultaneously affecting adjacent epithelial and mesenchymal cells or may result from a collision of coincidentally arising carcinoma and sarcoma. A tumour within the scope of this definition may be a primary or secondary tumour, whereby primary indicates that the tumour originated in the tissue where it is found rather than was established as a secondary site through metastasis from another tumour lesion. As outlined above, tumours within the scope of this definition may be benign or malign and may affect all anatomical structures of the body of a subject, preferably a mammal. For example, they comprise tumours of a) the bone marrow and bone marrow derived cells (leukemias), b) the endocrine and exocrine glands like e. g. thyroid, parathyroid, pituitary, adrenal glands, salivary glands, pancreas, c) the breast, like e. g. benign or malignant tumours in the mammary glands of either a male or a female, the mammary ducts, adenocarcinoma, medullary carcinoma, comedo carcinoma, Paget's disease of the nipple, inflammatory carcinoma of the young woman, d) the lung, e) the stomach, f) the liver and spleen, g) the small intestine, h) the colon, i) the bone and its supportive and connective tissues like malignant or benign bone tumour, e. g. malignant osteogenic sarcoma, benign osteoma, cartilage tumours; like malignant chondrosarcoma or benign chondroma; bone marrow tumours like malignant myeloma or benign eosinophilic granuloma, as well as metastatic tumours from bone tissues at other locations of the body; k) the mouth, throat, larynx, and the esophagus, l) the urinary bladder and the internal and external organs and structures of the urogenital system of male and female like ovaries, uterus, cervix of the uterus, testes, and prostate gland, m) the prostate, n) the pancreas, like ductal carcinoma of the pancreas; o) the lymphatic tissue like lymphomas and other tumours of lymphoid origin, p) the skin, q) cancers and tumour diseases of all anatomical structures belonging to the respiration and respiratory systems including thoracal muscles and linings, r) primary or secondary cancer of the lymph nodes s) the tongue and of the bony structures of the hard palate or sinuses, t) the mouth, cheeks, neck and salivary glands, u) the blood vessels including the heart and their linings, v) the smooth or skeletal muscles and their ligaments and linings, w) the peripheral, the autonomous, the central nervous system including the cerebellum, x) the adipose tissue. The tumours may be in any stage of their development such as early stages, where the tumour is still confined to the tissue of origin (or primary site), or when the cancer cells have already invaded adjacent tissue or distant parts of the body from the primary site, i.e. the tumour already metastasises.

The term “metastasis” in the context of tumours is well-known in the art. Metastasis of tumours is the process in which cancer cells can detach from the primary tumour and produce daughter tumours after spreading via blood or lymphatic vessels. As shown in FIG. 1, this includes the steps of intravasation, transit, arrest and adhesion the endothelium, extravasation, adhesion at the site of extravasation (seeding) and survival and growth of the metastasising tumour cells at the new site. Tumours which have a high incidence of metastasis are e.g. tumours of the lung, breast, colon, kidney, prostate, pancreas, cervix, as well as melanoma. Upon migration within an organ's cavity, tumour cells may also need to overcome a cellular (non-endothelial) barrier to enter the tissue underneath. This can involve the induction of necroptosis in (some of) the cells forming the barrier. Thus, it is envisaged that also metastases which do not pass through a blood or lymphatic vessel are inhibited using a necroptosis inhibitor. Preferably, in accordance with the present invention the necroptosis inhibitor is for use in preventing hematogenous and/or lymphatic metastasis, i.e. metastases which have passed through a blood or lymphatic vessel.

The term “prevent” in the context of tumour metastasis means that a tumour is precluded from metastasising in the presence of the inhibitor of the invention. On the molecular level the prevention is characterised in that the tumour cells do not cause necroptosis of endothelial cells to the extent that tumour cell extravasation occurs. As will be understood, even a reduction in the risk and/or rate of metastasis occurring will be beneficial as it may render the disease more manageable from a clinical standpoint and may increase the life expectancy of the patient afflicted with a tumour, in particular with regard to tumours characterised by a high rate and/or risk to metastasise. Therefore, the term “prevent” also encompasses the meaning of reducing the risk and/or rate of tumour metastasis. In other words, while the term “prevent” thus encompasses abolition of metastasis, i.e. no metastasis occurs, it also relates to a decrease in the risk and/or rate of metastasis of a tumour occurring, e.g. by at least (for each value) 30%, 40%, or 50% such as at least (for each value) 55%, 60%, 65%, 70% or 75%, preferred by at least (for each value) 80% or 85%, more preferred at least (for each value) 90% or 95% and most preferred by 100%, i.e. abolition of tumour metastasis, as mentioned before.

It is understood in accordance with the invention that the inhibitors as defined herein can be used for the prevention of metastasis of tumours that have not yet begun to metastasise as well as those that already metastasise.

Preferably, the inhibitor of necroptosis is for use in preventing metastasis of tumours in a mammalian subject. More preferably, the subject is human.

In accordance with the present invention, it was surprisingly found that endothelial transmigration of tumour cells involves necroptosis of endothelial cells. Without wishing to be bound to a scientific theory, it is thought that metastasising tumour cells can induce necroptosis in endothelial cells. This contributes to the transmigration of the tumour cells through the endothelium and thus to the extravasation of the tumour cells. This important step of metastasis formation can be prevented by blocking necroptosis using a necroptosis inhibitor.

These findings are surprising for a number of reasons. The present dogma of tumour cell extravasation suggests that, similar to leukocytes, tumour cells transmigrate the endothelium by inducing endothelial cell retraction via cellular rearrangements without significantly disrupting the endothelial monolayer (Reymond et al., 2013). Further, even though induction of cell death in endothelial cells was proposed as an alternative mechanism contributing to transmigration this cell death was described to be exclusively apoptotic. On this background the use of a necroptosis inhibitor is counter-intuitive especially because compounds inducing cell death, specifically apoptosis, are used for the treatment of tumours.

Thus, the inventors to their best knowledge have revealed for the first time a direct link between the necroptosis pathway and the transmigration of tumour cells through the endothelium.

Apoptosis is well known to play a role in many physiological processes. On the other hand, few physiological functions are known for necroptosis. Perhaps the best evidenced role for necroptosis in vivo is in the regulation of viral infection (Cho et al., 2009). Given the so far limited in-vivo functions reported for necroptosis it is surprising that this type of cell death is involved in transmigration of metastasising tumour cells trough the endothelium and that necroptosis inhibitors can be used to inhibit this process and thus the overall process of metastasis formation.

The present invention provides advantageous treatment opportunities because, according to the current knowledge, necroptosis inhibitors can be expected to be associated with fewer side effects as compared to apoptosis inhibitors due to the limited functions of necroptosis in normal physiology. Apoptosis inhibitors would also block apoptosis in situations where this type of death is required to maintain normal physiology and would therefore potentially be associated with severe side effects especially when applied systemically. In particular, if a tumour already exists or is expected to develop, the application of an apoptosis inhibitor is likely to be harmful rather than beneficial. This is because apoptosis is a mechanism to keep abnormal and potentially cancerogenous cells in check and counteracting this control mechanism may enhance tumour development and growth. Thus, the use of necroptosis inhibitors in accordance with the present invention provides an opportunity to prevent transmigration of metastasising tumour cells through the endothelium which, according to the current knowledge, is not expected to be associated with severe side effects.

Further the invention relates to a method of preventing the metastasis of tumours by inhibiting necroptosis comprising administering a pharmaceutically effective amount of an inhibitor of necroptosis to a subject in need thereof.

The definitions and preferred embodiments described for the inhibitor for use according to the present invention apply mutatis mutandis also to the method of preventing metastasis of tumours.

As used herein the term “subject in need thereof” relates to an animal, preferably a vertebrate, more preferably a mammal, and most preferably to a human subject which suffers from a tumour.

The invention further relates to a method for modulating the transmigration of metastasising tumour cells through endothelium by modulating necroptosis, comprising the steps of: a) contacting endothelium with a modulator of necroptosis; and b) providing metastasising tumour cells and allowing said metastasising tumour cells to transmigrate through the endothelium.

The definitions and preferred embodiments described for the inhibitor for use according to the present invention and for the method of preventing metastasis of tumours apply mutatis mutandis also to the method for modulating the transmigration of metastasising tumour cells through endothelium.

Preferably, the method for modulating the transmigration of metastasising tumour cells through endothelium is an in-vitro or an ex-vivo method. Performing the method of the invention in test animals, for example for research purposes, is also envisaged. The test animal is not a human.

As used herein, the term “modulating” refers to altering the rate of transmigration and encompasses both enhancing as well as preventing transmigration.

The term “transmigration” as used herein refers to the process of cells moving through a layer of other cells. Thus, the term “transmigration through endothelium” refers to the process of cells crossing from one side of an endothelial cell layer to the other side thereof. In vivo, this encompasses cells moving from the inside of the vessel, through the layer of endothelial cells, to the subendothelial matrix and finally into the surrounding tissue.

As used herein the term “endothelium” refers to a layer of endothelial cells that lines the interior surface of blood vessels or lymphatic vessels. Also encompassed are endothelial cells which form a continuous layer in vitro and which mimic the natural endothelium.

The term “comprising” in the context of the methods of the invention denotes that the methods may include further steps. Alternatively, the methods consists of the specified steps, i.e. no other steps are performed in addition to the steps described herein.

The term “contacting endothelium with a modulator of necroptosis” used herein refers to bringing the endothelial cells and the modulator of necroptosis into such close proximity and under such conditions that an interaction between the modulator and the cells can take place. In particular, if the modulator affects necroptosis by acting on an intracellular compound, the conditions are chosen in a manner that allows for the modulator to be taken up by the cells of the endothelium. At the same time the term encompasses that the conditions such as temperature, pH, concentration of the modulator under which the contact occurs as well as the concentration of the modulator are such that the contact is not toxic or harmful in another way to the cells on its own, i.e. in the absence of the metastasising tumour cells.

As used herein, a “modulator” is a compound which either enhances or prevents/slows down the process or which enhances or reduces the activity of the protein. In other words, the modulator is an activator/enhancer or an inhibitor. Therefore, a modulator that inhibits necroptosis is an inhibitor of necroptosis as defined herein above. A modulator activating the transmigration of metastasising tumour cells through endothelium may be useful, for example, for research purposes. For example, a modulator acting as an enhancer of tumour cell migration may be used in an in-vitro assay or in vivo in a test animal for identifying (particularly potent) necroptosis inhibitors, i.e. compounds which can inhibit necroptosis and/or tumour cell transmigration even in the presence of the modulator. This may mimic a situation where endothelial cells are subject to an alteration, such as e.g. a mutation, rendering them more susceptible to necroptosis. A modulator enhancing necroptosis may also be useful in studying necroptosis, for example for research purposes, in a setting where the number of necroptotic cells would usually be limited. In such a setting a modulator increasing necroptosis may be employed to enhance the number of necroptotic cells and hence to facilitate the analysis.

As used herein the term “providing metastasising tumour cells” refers to actively adding metastasising tumour cells directly or indirectly to the endothelium. Indirectly adding metastasising tumour cells to the endothelium encompasses, for example, the active generation of a primary tumour in a test animal, for example, by the application of tumourigenic cells or a cancerogenous substance to the test animal provided that the generated primary tumour metastasises and provided that the tumourigenic cells are not directly added to the endothelium, for example, by injection into the blood stream. It is preferred that metastasising tumour cells are not provided by inducing the formation of a primary tumour. Providing the metastasising tumour cells directly to the endothelium is preferred. The method of the invention is not a method of treatment by therapy or surgery practised on the human or animal body. The addition of the metastasising tumour cells may be performed prior to, at the same time as or after the addition of the modulator of necroptosis, i.e. steps a) and b) may be performed at the same time or step a) may be performed before step b) or after step b). It is understood that in case the modulator of necroptosis is added after the metastasising tumour cells, i.e. step a) is performed after step b), the time difference is such that tumour cell transmigration has not yet occurred before the modulator is added. On the other hand, if the modulator of necroptosis is added before the metastasising tumour cells, the time span between the additions is not so large that in the meantime the modulator has been degraded or inactivated in a different manner. The order of steps and the time frame depends on the experimental setting. If necroptosis and transmigration are expected to occur quickly, for example, in an in-vitro assay or if the tumour cells are intravenously injected to a test animal, the modulator should be applied, before, simultaneously with or immediately after the metastasising tumour cells. Preferably, the modulator of necroptosis is applied before the metastasising tumour cells in that case, i.e. step a) is performed before b). On the other hand, if induction of necroptosis and transmigration are expected to occur not immediately after addition of the tumour cells, e.g., if the tumour cells are applied to a test animal where they first need to form a primary tumour which then gives rise to the metastasising tumour cells which in turn need to separate from the primary tumour and start the process of metastasis before necroptosis induction and transmigration occur, the modulator is preferably applied after the tumour cells, i.e. around the time when induction of necroptosis and transmigration are expected. In that case, it is preferred that step a) is performed after step b).

The term “allowing said metastasising tumour cells to transmigrate through the endothelium” means that the conditions and the time of contact between the endothelium and the tumour cells are selected in a manner that would allow the metastasising tumour cells to cross from one side of the endothelial cell layer to the other side thereof in the absence of the modulator of necroptosis. In case the modulator of necroptosis is an inhibitor of necroptosis it is understood that the term “allowing” also encompasses that the metastasising tumour cells are prevented from transmigrating the endothelium by this inhibitor, i.e. in the presence of the necroptosis inhibitor the metastasising tumour cells may be unable to cross from one side of the endothelial cell layer to the other side thereof albeit conditions and time are selected such that transmigration would occur in the absence of said inhibitor. In other words, the term does not require that the metastasising tumour cells actually transmigrate through the endothelium. Instead, it is sufficient that the metastasising tumour cells could transmigrate through the endothelium, if not prevented from doing so by the modulator of necroptosis.

In addition, the invention relates to an in-vitro method of identifying an inhibitor of necroptosis suitable as a lead compound and/or as a medicament for the prevention of tumour metastasis, comprising the steps of: a) allowing metastasising tumour cells to transmigrate the endothelium i) in the presence of a test agent and ii) in the absence of said test agent; b) determining the level of metastasising tumour cell transmigration through the endothelium and/or the level of endothelial cell necroptosis for a)i) and a)ii); c) comparing the level(s) determined in step b) for a)i) with the level(s) determined in step b) for a)ii), wherein a decrease in the level(s) for a)i) as compared to the level(s) of a)ii) is indicative for the test agent to be an inhibitor of necroptosis suitable as a lead compound and/or as a medicament for the prevention of tumour metastasis.

The term “lead compound” as used herein refers to a compound that can be classified as necroptosis inhibitor as defined herein above. Such lead compounds are used as starting compounds for developing drugs to prevent the metastasis of tumours in that they may be optimised with regard to, e.g., their potency, their pharmacokinetic profile or their suitability to be used in a certain pharmaceutical formulation, so as to arrive at a compound which may be, advantageously, safely and effectively used in a pharmaceutical composition. Methods and tools for the optimisation of the pharmacological properties of compounds identified in screens, the lead compounds, are known in the art. For example, in silico tools for optimizing lead compounds are known in the art and described, e.g., in Cruciani et al., European Journal of Pharmaceutical Sciences, vol. 11, suppl. 2, p. S29-S39 (2000). Furthermore, high-throughput approaches for evaluating properties of lead compounds have been described in Tarbit and Berman, Current Opinion in Chemical Biology, vol. 2, issue 3, p. 411-416 (1998).

In a more preferred embodiment of the methods of the invention, the optimisation comprises modifying necroptosis inhibitor to achieve: i) modified spectrum of activity, organ specificity, and/or ii) improved potency, and/or iii) decreased toxicity (improved therapeutic index), and/or iv) decreased side effects, and/or v) modified onset of therapeutic action, duration of effect, and/or vi) modified pharmacokinetic parameters (resorption, distribution, uptake into a specific cell type, metabolism and excretion), and/or vii) modified physico-chemical parameters (solubility, hygroscopicity, colour, taste, odour, stability, state), and/or viii) improved general specificity, organ/tissue specificity, and/or ix) optimised application form and route by (a) esterification of carboxyl groups, or (b) esterification of hydroxyl groups with carboxylic acids, or (c) esterification of hydroxyl groups to, e.g. phosphates, pyrophosphates or sulfates or hemi-succinates, or (d) formation of pharmaceutically acceptable salts, or (e) formation of pharmaceutically acceptable complexes, or (f) synthesis of pharmacologically active polymers, or (g) introduction of hydrophilic moieties, or (h) introduction/exchange of substituents on aromates or side chains, change of substituent pattern, or (i) modification by introduction of isosteric or bioisosteric moieties, or (j) synthesis of homologous compounds, or (k) introduction of branched side chains, or (l) conversion of alkyl substituents to cyclic analogues, or (m) derivatisation of hydroxyl groups to ketales, acetales, or (n) N-acetylation to amides, phenylcarbamates, or (o) synthesis of Mannich bases, imines, or (p) transformation of ketones or aldehydes to Schiffs bases, oximes, acetales, ketales, enolesters, oxazolidines, thiazolidines or combinations thereof.

The various steps recited above are generally known in the art, as described above. They include or rely on quantitative structure-activity relationship (QSAR) analyses (Kubinyi (1992) “Hausch-Analysis and Related Approaches”, VCH Verlag, Weinheim), combinatorial biochemistry, classical chemistry and others (see, for example, Holzgrabe and Bechtold (2000) Deutsche Apotheker Zeitung 140(8), 813).

As used herein the term “test agent” refers to a substance that is or is to be analysed for its ability to inhibit necroptosis. The test agent may be a compound that is designed or expected to target a component of the necroptosis pathway and in particular a specific component of the necrosome. However, any substance may be employed as a test agent, i.e. also substances which are not specifically designed to target a particular component of the necroptosis pathway.

It is understood that steps a)i) and a)ii) may be performed in any order, i.e. step a)i) may be performed before, after or simultaneously with step a)ii). Similarly, in step b) the level of metastasising tumour cell transmigration through the endothelium in the presence (a)i)) of the test agent may be determined before, after or simultaneously with determining the level of metastasising tumour cell transmigration through the endothelium in the absence (a)ii)) of the test agent. The same applies with regard to determining the level of endothelial cell necroptosis in the presence or absence of the test agent. Further, in step b) determining the levels of metastasising tumour cell transmigration through the endothelium and/or endothelial cell necroptosis in the absence of the test agent (a)ii)) does not have to be performed experimentally if data regarding the transmigration of said metastasising tumour cells through said endothelium in the absence of a test agent and/or regarding the level of endothelial cell necroptosis under the same experimental conditions have already been obtained in earlier experiments or are known from another source. In other words, in step c) the data determined in step b) for a)i) may be compared also to pre-existing data for a)ii). Such pre-existing data may be, for example, a statistical readout of transmigration and/or necroptotic cell death obtained from control experiments, i.e. experiments carried out in the absence of a test agent, previously carried out. A statistical readout is in general obtained by weighting the results from repeating the same control experiment or by weighting the results from different control experiments. Statistical methods for obtaining a statistical readout are well known in the art.

As used herein the term “determining the level of metastasising tumour cell transmigration through the endothelium” refers to analysing and quantifying how many metastasising tumour cells have passed from one side of the endothelial cell layer to the other side of this layer. Methods for analysing tumour cell transmigration are known in the art and include, without limitation, the following methods. For example, fluorescently labelled tumour cells (GFP-expression or pre-staining with fluorescent dyes such as Calcein-AM) may be allowed to transmigrate over an endothelial layer that grows on a transwell plate (pore size e.g. 8 μm). After a certain time which depends on the tumour cell used, but which is usually between 6 -24 h, the non-transmigrated tumour cells on the top of the filter are removed and the number of the transmigrated tumour cells on the lower side of the filter or on the bottom of the receiver well can be recorded with a microscope and further analysed and quantified. Alternatively, unlabelled tumour cells may be allowed to transmigrate over the endothelial layer. The tumour cells can then by stained after transmigration by any commonly used cell dye, imaged, analysed and quantified.

As used herein the term “determining the level of endothelial cell necroptosis” refers to quantifying how many endothelial cells have died by necroptosis. In other word, the term encompasses differentiating between living cells and dead cells, further distinguishing within the dead cells by which type of cell death these cells have died and, thus, determining the number of cells that have died by necroptosis. The latter step requires, in particular, that necroptotic/necrotic cells are distinguished from cells that have died by another form of cell death such as apoptosis. Preferably, the level of endothelial cell necroptosis is determined using the methods described in the Examples herein below.

Methods for comparing data obtained in the presence of a test agent to data obtained in the absence of a test compound are well known in the art. For example, the fold-change can be calculated by dividing the level in the presence of the test agent (a)i)) by the corresponding level in the absence of the test agent (a)ii)). If the ratio of the level for a)i) to the level of a)ii) is smaller than 0.8, preferably, smaller than 0.7, smaller than 0.6, smaller than 0.5, preferably smaller than 0.25, more preferably, smaller than 0.1 and even more preferably smaller than 0.05 such as 0.02 or even 0, then test agent is considered to be an inhibitor of necroptosis in accordance with the present invention.

In a preferred embodiment of the inhibitor of the invention or the methods of the invention, the inhibitor, modulator or test agent is an antibody, an antibody mimetic, a dominant negative protein, a siRNA, a shRNA, a miRNA, a ribozyme, an aptamer, an antisense nucleic acid molecule, a small molecule or a modified version of these inhibitors, modulators or test agents.

The term “antibody” as used herein comprises, for example, polyclonal or monoclonal antibodies. Furthermore, also derivatives or fragments thereof, which still retain the binding specificity, are comprised in the term “antibody”. Antibody fragments or derivatives comprise, inter alia, Fab or Fab′ fragments, Fd, F(ab′)₂, Fv or scFv fragments, single domain V_{H} or V-like domains, such as VhH or V-NAR-domains, as well as multimeric formats such as minibodies, diabodies, tribodies or triplebodies, tetrabodies or chemically conjugated Fab′-multimers (see, for example, Altshuler E P, Serebryanaya D V, Katrukha A G. 2010, Biochemistry (Mosc)., vol. 75(13), 1584 Holliger P, Hudson P J. 2005, Nat Biotechnol., vol. 23(9), 1126). The term “antibody” also includes embodiments such as chimeric (human constant domain, non-human variable domain), single chain and humanised (human antibody with the exception of non-human CDRs) antibodies.

Various techniques for the production of antibodies are well known in the art and described, e.g. in Altshuler et al., 2010 (Altshuler EP, Serebryanaya DV, Katrukha AG. 2010, Biochemistry (Mosc)., vol. 75(13), 1584). Thus, polyclonal antibodies can be obtained from the blood of an animal following immunisation with an antigen in mixture with additives and adjuvants and monoclonal antibodies can be produced by any technique which provides antibodies produced by continuous cell line cultures. Examples for such techniques are described, e.g. in Harlow E and Lane D, Cold Spring Harbor Laboratory Press, 1988; Harlow E and Lane D, Using Antibodies: A Laboratory Manual, Cold Spring Harbor Laboratory Press, 1999 and include the hybridoma technique originally described by Kohler and Milstein, 1975, the trioma technique, the human B-cell hybridoma technique (see e.g. Kozbor D, 1983, Immunology Today, vol.4, 7; Li J, et al. 2006, PNAS, vol. 103(10), 3557) and the EBV-hybridoma technique to produce human monoclonal antibodies (Cole et al., 1985, Alan R. Liss, Inc, 77-96). Furthermore, recombinant antibodies may be obtained from monoclonal antibodies or can be prepared de novo using various display methods such as phage, ribosomal, mRNA, or cell display. A suitable system for the expression of the recombinant (humanised) antibodies may be selected from, for example, bacteria, yeast, insects, mammalian cell lines or transgenic animals or plants (see, e.g., U.S. Pat. No. 6,080,560; Holliger P, Hudson P J. 2005, Nat Biotechnol., vol. 23(9), 11265). Further, techniques described for the production of single chain antibodies (see, inter alia, U.S. Pat. No. 4,946,778) can be adapted to produce single chain antibodies specific for an epitope of a component of the necroptosis pathway. Surface plasmon resonance as employed in the BIAcore system can be used to increase the efficiency of phage antibodies.

As used herein the term “antibody mimetics” refers to compounds which, like antibodies, can specifically bind antigens, but which are not structurally related to antibodies. Antibody mimetics are usually artificial peptides or proteins with a molar mass of about 3 to 20 kDa.

For example, an antibody mimetic may be selected from the group consisting of affibodies (which are based on the Z-domain of staphylococcal protein A) adnectins (based on the tenth domain of human fibronectin), anticalins (derived from lipocalins), DARPins (derived from ankyrin repeat proteins), avimers (based e.g. on multimerised Low Density Lipoprotein Receptor (LDLR)-A), nanofitins (derived from the DNA binding protein Sac7d of Sulfolobus acidocaldarius), affilins (structurally derived from gamma-B crystalline or ubiquitin), Kunitz domain peptides (derived from the Kunitz domains of various protease inhibitors) and Fynomers® which are derived from the human Fyn SH3 domain (see e.g. Grabulovski et al. (2007) JBC, 282, p. 3196-3204, WO 2008/022759, Bertschinger et al (2007) Protein Eng Des Sel 20(2):57-68, Gebauer and Skerra (2009) Curr Opinion in Chemical Biology 13:245-255, or Schlatter et al. (2012), MAbs 4:4, 1-12). These polypeptides are well known in the art and will be described in further detail herein below.

As used herein, the term “DARPin” refers to a designed ankyrin repeat domain (166 residues), which provides a rigid interface arising from typically three repeated 13-turns. DARPins usually carry three repeats corresponding to an artificial consensus sequence, whereby six positions per repeat are randomised. Consequently, DARPins lack structural flexibility (Gebauer and Skerra, 2009).

A “nanofitin” (also known as affitin) is an antibody mimetic protein that is derived from the DNA binding protein Sac7d of Sulfolobus acidocaldarius. Nanofitins usually have a molecular weight of around 7kDa and are designed to specifically bind a target molecule by randomising the amino acids on the binding surface (Mouratou B, Behar G, Paillard-Laurance L, Colinet S, Pecorari F., (2012) Methods Mol Biol.; 805:315-31).

The term “anticalin” as used herein refers to an engineered protein derived from a lipocalin (Beste G, Schmidt FS, Stibora T, Skerra A. (1999) Proc Natl Acad Sci U S A. 96(5):1898-903; Gebauer and Skerra (2009) Curr Opinion in Chemical Biology 13:245-255). Anticalins possess an eight-stranded 6-barrel which forms a highly conserved core unit among the lipocalins and naturally forms binding sites for ligands by means of four structurally variable loops at the open end. Anticalins, although not homologous to the IgG superfamily, show features that so far have been considered typical for the binding sites of antibodies: (i) high structural plasticity as a consequence of sequence variation and (ii) elevated conformational flexibility, allowing induced fit to targets with differing shape.

An “adnectin” (also referred to as “monobody”) as used herein, is based on the 10th extracellular domain of human fibronectin III (10Fn3), which adopts an Ig-like β-sandwich fold of 94 residues with 2 to 3 exposed loops, but lacks the central disulphide bridge (Gebauer and Skerra (2009) Curr Opinion in Chemical Biology 13:245-255). Adnectins with the desired target specificity can be genetically engineered by introducing modifications in specific loops of the protein.

The term “affibody” as used herein refers to a family of antibody mimetics which is derived from the Z-domain of staphylococcal protein A. Structurally, affibody molecules are based on a three-helix bundle domain which can also be incorporated into fusion proteins. In itself, an affibody has a molecular mass of around 6kDa and is stable at high temperatures and under acidic or alkaline conditions. Target specificity is obtained by randomisation of 13 amino acids located in two alpha-helices involved in the binding activity of the parent protein domain (Feldwisch J, Tolmachev V.; (2012) Methods Mol Biol. 899:103-26).

The term “affilin” as used herein refers to antibody mimetics that are developed by using either gamma-B crystalline or ubiquitin as a scaffold and modifying amino-acids on the surface of these proteins by random mutagenesis. Selection of affilins with the desired target specificity is effected, for example, by phage display or ribosome display techniques. Depending on the scaffold, affilins have a molecular weight of approximately 10 or 20kDa. As used herein the term affilin also refers to di- or multimerised forms of affilins (Weidle U H, et al., (2013), Cancer Genomics Proteomics;10(4):155-68).

The term “avimer” as used herein refers to a class of antibody mimetics which consist of two or more peptide sequences of 30 to 35 amino acids each which are derived from A-domains of various membrane receptors and which are connected by linker peptides. Binding of target molecules occurs via the A-domain and domains with the desired binding specificity can, for example, be selected by phage display techniques. Binding specificity of the different A-domains contained in an avimer may, but does not have to, be identical (Weidle U H, et al., (2013), Cancer Genomics Proteomics;10(4):155-68).

A “Kunitz domain peptide” is derived from the Kunitz domain of a Kunitz-type protease inhibitor such as bovine pancreatic trypsin inhibitor (BPTI), amyloid precursor protein (APP) or tissue factor pathway inhibitor (TFPI). Kunitz domains have a molecular weight of approximately 6 kDA and domains with the required target specificity can be selected by display techniques such as phage display (Weidle et al., (2013), Cancer Genomics Proteomics;10(4):155-68).

As used herein the term “Fynomer®” refers to a non-immunoglobulin-derived binding polypeptide derived from the human Fyn SH3 domain. Fyn SH3-derived polypeptides are well-known in the art and have been described e.g. in Grabulovski et al. (2007) JBC, 282, p. 3196-3204, WO 2008/022759, Bertschinger et al (2007) Protein Eng Des Sel 20(2):57-68, Gebauer and Skerra (2009) Curr Opinion in Chemical Biology 13:245-255, or Schlatter et al. (2012), MAbs 4:4, 1-12).

The term “dominant negative protein” as used herein refers to versions of pro-necroptotic proteins which maintain the ability to interact with the interaction partners of the corresponding naturally occurring protein but which lack at least the pro-necroptotic activity of the corresponding naturally occurring protein. Thus, a dominant negative protein is a protein competing with the corresponding naturally occurring protein for interaction partners but lacking the pro-necroptotic activity. Examples of dominant negative proteins are inactive variants of proteins effecting necroptosis. This includes soluble variants of the death receptor lacking the transmembrane domain. These receptor-like proteins can still bind to the natural ligand thus preventing its binding to the transmembrane receptors. At the same time the soluble receptor cannot pass on the signal into the cell and thus cannot induce necroptosis. It is understood that also membrane-anchored and soluble receptors can act as a dominant negative protein as long as they can bind to the natural ligand and are unable to intracellularly transmit the signal. This also encompasses soluble forms of receptors generated by cleavage of the extracellular domain, naturally occurring alternative forms of a receptor or other receptors binding to the same endogenous ligand, thus preventing its binding to the receptor to be inhibited, but being unable to transmit the signal which the ligand would induce upon binding to the receptor to be inhibited. Such receptors are also referred to as decoy-receptors. As used herein the term “decoy receptor” also encompasses naturally occurring receptors. Preferably, the dominant negative protein corresponds to a specific component of the necrosome, i.e. a component of the necrosome that is not required in other signalling pathways in particular in the induction of apoptosis. It may be, for example, a kinase-dead variant of RIPK1 or RIPK3. In the case of dominant negative versions of intracellular proteins it is preferred that these are employed in the form of a nucleic acid molecule encoding the dominant negative protein.

As used herein, the term “small interfering RNA (siRNA)”, also known as short interfering RNA or silencing RNA, refers to a class of 18 to 30, preferably 19 to 25, most preferred 21 to 23 or even more preferably 21 nucleotide-long double-stranded RNA molecules that play a variety of roles in biology. Most notably, siRNA is involved in the RNA interference (RNAi) pathway where the siRNA interferes with the expression of a specific gene. In addition to their role in the RNAi pathway, siRNAs also act in RNAi-related pathways, e.g. as an antiviral mechanism or in shaping the chromatin structure of a genome.

siRNAs naturally found in nature have a well defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest. Essentially any gene of which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, it is preferred that at least one RNA strand has a 5′- and/or 3′-overhang. Preferably, one end of the double-strand has a 3′-overhang from 1-5 nucleotides, more preferably from 1-3 nucleotides and most preferably 2 nucleotides. The other end may be blunt-ended or has up to 6 nucleotides 3′-overhang. In general, any RNA molecule suitable to act as siRNA is envisioned in the present invention. The most efficient silencing was so far obtained with siRNA duplexes composed of 21-nt sense and 21-nt antisense strands, paired in a manner to have a 2-nt 3′-overhang. The sequence of the 2-nt 3′ overhang makes a small contribution to the specificity of target recognition restricted to the unpaired nucleotide adjacent to the first base pair (Elbashir et al. 2001). 2′-deoxynucleotides in the 3′ overhangs are as efficient as ribonucleotides, but are often cheaper to synthesize and probably more nuclease resistant. Delivery of siRNA may be accomplished using any of the methods known in the art, for example by combining the siRNA with saline and administering the combination intravenously or intranasally or by formulating siRNA in glucose (such as for example 5% glucose) or cationic lipids and polymers can be used for siRNA delivery in vivo through systemic routes either intravenously (IV) or intraperitoneally (IP) (Fougerolles et al. (2008), Current Opinion in Pharmacology, 8:280-285; Lu et al. (2008), Methods in Molecular Biology, vol. 437: Drug Delivery Systems Chapter 3: Delivering Small Interfering RNA for Novel Therapeutics). The activity and specificity of siRNAs can be altered by various modifications such as by inclusion of a blocking group at the 3′ and 5′ ends, wherein the term “blocking group” refers to substituents of that can be attached to oligonucleotides or nucleomonomers, either as protecting groups or coupling groups for synthesis (cf. WO 98/13526, EP 2221377 B1), by inclusion of agents that enhance the affinity to the target sequence such as intercalating agents (e.g., acridine, chlorambucil, phenazinium, benzophenanthirdine), attaching a conjugating or complexing agent or encapsulating it to facilitate cellular uptake, or attaching targeting moieties for targeted delivery.

A short hairpin RNA (shRNA) is a sequence of RNA that makes a tight hairpin turn that can be used to silence gene expression via RNA interference. Experimentally, shRNA uses a vector introduced into cells and utilises the U6 promoter to ensure that the shRNA is expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it.

si/shRNAs to be used in the present invention are preferably chemically synthesized using appropriately protected ribonucleoside phosphoramidites and a conventional DNA/RNA synthesizer. Suppliers of RNA synthesis reagents are Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). Most conveniently, siRNAs or shRNAs are obtained from commercial RNA oligo synthesis suppliers, which sell RNA-synthesis products of different quality and costs. In general, the RNAs applicable in the present invention are conventionally synthesized and are readily provided in a quality suitable for RNAi.

Further molecules effecting RNAi include, for example, microRNAs (miRNA). Said RNA species are single-stranded RNA molecules which, as endogenous RNA molecules, regulate gene expression. Binding to a complementary mRNA transcript triggers the degradation of said mRNA transcript through a process similar to RNA interference.

A ribozyme (from ribonucleic acid enzyme, also called RNA enzyme or catalytic RNA) is an RNA molecule that catalyzes a chemical reaction. Many natural ribozymes catalyze either their own cleavage or the cleavage of other RNAs, but they have also been found to catalyze the aminotransferase activity of the ribosome. Non-limiting examples of well-characterised small self-cleaving RNAs are the hammerhead, hairpin, hepatitis delta virus, and in vitro-selected lead-dependent ribozymes, whereas the group I intron is an example for larger ribozymes. The principle of catalytic self-cleavage is well established in the art. The hammerhead ribozymes are characterised best among the RNA molecules with ribozyme activity. Since it was shown that hammerhead structures can be integrated into heterologous RNA sequences and that ribozyme activity can thereby be transferred to these molecules, it appears that catalytic antisense sequences for almost any target sequence can be created, provided the target sequence contains a potential matching cleavage site. The basic principle of constructing hammerhead ribozymes is as follows: An interesting region of the RNA, which contains the GUC (or CUC) triplet, is selected. Two oligonucleotide strands, each usually with 6 to 8 nucleotides, are taken and the catalytic hammerhead sequence is inserted between them. Molecules of this type were synthesized for numerous target sequences. They showed catalytic activity in vitro and in some cases also in vivo. The best results are usually obtained with short ribozymes and target sequences.

Aptamers are nucleic acid molecules or peptide molecules that bind a specific target molecule. Aptamers are usually created by selecting them from a large random sequence pool, but natural aptamers also exist in riboswitches. Aptamers can be used for both basic research and clinical purposes as macromolecular drugs. Aptamers can be combined with ribozymes to self-cleave in the presence of their target molecule. These compound molecules have additional research, industrial and clinical applications (Osborne et. al. (1997), Current Opinion in Chemical Biology, 1:5-9; Stull & Szoka (1995), Pharmaceutical Research, 12, 4:465-483).

More specifically, aptamers can be classified as nucleic acid aptamers, such as DNA or RNA aptamers, or peptide aptamers. Whereas the former normally consist of (usually short) strands of oligonucleotides, the latter preferably consist of a short variable peptide domain, attached at both ends to a protein scaffold.

Nucleic acid aptamers are nucleic acid species that, as a rule, have been engineered through repeated rounds of in vitro selection or equivalently, SELEX (systematic evolution of ligands by exponential enrichment) to bind to various molecular targets such as small molecules, proteins, nucleic acids, and even cells, tissues and organisms.

Peptide aptamers usually are peptides or proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range). The variable peptide loop typically comprises 10 to 20 amino acids, and the scaffold may be any protein having good solubility properties. Currently, the bacterial protein Thioredoxin-A is the most commonly used scaffold protein, the variable peptide loop being inserted within the redox-active site, which is a -Cys-Gly-Pro-Cys-loop in the wild protein, the two cysteins lateral chains being able to form a disulfide bridge. Peptide aptamer selection can be made using different systems, but the most widely used is currently the yeast two-hybrid system.

Aptamers offer the utility for biotechnological and therapeutic applications as they offer molecular recognition properties that rival those of the commonly used biomolecules, in particular antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. Non-modified aptamers are cleared rapidly from the bloodstream, with a half-life of minutes to hours, mainly due to nuclease degradation and clearance from the body by the kidneys, a result of the aptamer's inherently low molecular weight. Unmodified aptamer applications currently focus on treating transient conditions such as blood clotting, or treating organs such as the eye where local delivery is possible. This rapid clearance can be an advantage in applications such as in vivo diagnostic imaging. Several modifications, such as 2′-fluorine-substituted pyrimidines, polyethylene glycol (PEG) linkage, fusion to albumin or other half life extending proteins etc. are available such that the half-life of aptamers can be increased for several days or even weeks.

Also useful is the combination of an aptamer recognising a small compound with a hammerhead ribozyme. The conformational change induced in the aptamer upon binding the target molecule is supposed to regulate the catalytic function of the ribozyme.

The term “antisense nucleic acid molecule” as used herein refers to a nucleic acid which is complementary to a target nucleic acid. An antisense molecule is capable of interacting with the target nucleic acid, more specifically it is capable of hybridising with the target nucleic acid. Due to the formation of the hybrid, transcription of the target gene(s) and/or translation of the target mRNA is reduced or blocked. Standard methods relating to antisense technology have been described (see, e.g., Melani et al., Cancer Res. (1991) 51:2897-2901).

A “small molecule” as used herein may be, for example, an organic small molecule. Organic small molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. Alternatively, the “small molecule” may be an inorganic compound. Inorganic compounds are derived from mineral sources and include all compounds without carbon atoms (except carbon dioxide, carbon monoxide and carbonates). Preferably, the small molecule has a molecular weight of less than about 2000 amu, or less than about 1000 amu such as less than about 500 amu, and even more preferably less than about 250 amu. The size of a small molecule can be determined by methods well-known in the art, e.g., mass spectrometry. The small molecules may be designed, for example, based on the crystal structure of the target molecule, where sites presumably responsible for the biological activity can be identified and verified in in-vivo assays such as in-vivo high-throughput screening (HTS) assays.

The term “modified versions of these inhibitors” as used herein refers to versions of the inhibitors which are optimized, e.g., by the methods described herein above in relation to lead compounds.

In a further preferred embodiment of the method of the invention, the subject or the endothelium is mammalian.

In addition, in another preferred embodiment of the inhibitor of the invention or the method of the invention, the inhibitor prevents or the modulator modulates the formation and/or the necroptosis-inducing activity of the necrosome.

As used herein the term “the formation of the necrosome” refers to the assembly of RIPK1, RIPK3, into a complex, to the recruitment of MLKL and, optionally, to the further assembly into a multiprotein complex comprising additional components such as TRADD, FADD, caspase 8, and/or PGAM5. Accordingly, an inhibitor preventing the formation of the necrosome interferes with the interaction of at least one of the necrosome's components with its binding partner within the necrosome. Preferably, the inhibitor reduces or abolishes the interaction between the components of the core complex, i.e. between RIPK1 and RIPK3. In this regard, for example, a peptide mimicking the RHIM domain of RIPK1 or RIPK3 which competes with the kinase whose RHIM it mimics for binding to the respectively other kinase is envisaged. Alternatively, an inhibitor may prevent the binding of MLKL to RIPK3, for example again by acting as a competitive inhibitor. A modulator modulating the formation of the necrosome may either act as an inhibitor or as an enhancer. In the latter case, the modulator may either enhance the interaction of at least one of the necrosome's components, preferably of one of the core components, with its binding partner within the necrosome or it may enhance the overall stability of the necrosome. As mentioned herein above, activating necroptosis and/or tumour cell transmigration may be useful for research purposes. Thus, a modulator enhancing, for example, the stability of the necrosome may, e.g., be used in an assay for the identification of (particularly potent) necroptosis inhibitors.

The term “the necroptosis-inducing activity of the necrosome” as used herein refers to the necrosome's ability to engage the effector mechanism of necroptosis, i.e. to the ability to induce downstream events that ultimately result in the death of the cell. This ability depends, for example, on the activities of the necrosome's components, such as for example on the kinase activities of RIPK1 and RIPK3. Also the phosphorylation of MLKL by RIPK3 (Sun et al., 2013, Cell 148, 213-227; Wang et al., 2014, Mol. Cell. 54, 133-146) and the ability of MLKL to multimerise via its N-terminal four helix bundle and to interact with phosphoinositol phosphates (PIPs) via positively charged residues within that helix bundle has been described to be required for necroptosis (Dondelinger et a., 2014, Cell Reports 7, 971-981). An inhibitor preventing the necroptosis-inducing activity of the necrosome may therefore be an inhibitor interfering with the activity of one of the necrosome's components which effects or contributes to engaging the effector mechanism of necroptosis. Preferably, the activity prevented by the inhibitor is an activity specifically required for necroptosis but not for another signalling pathway. If the modulator acts as an inhibitor, the above considerations with regard to the inhibitor apply also with regard to the modulator. If on the other hand the modulator acts as an enhancer, it may increase the enzymatic activity of at least one of the necrosome's components either directly, for example, by favouring and/or stabilising the active conformation of that component or indirectly by reducing an event, such as for example, a modification, that would otherwise reduce the component's activity.

In another preferred embodiment of the inhibitor of the invention or the method of the invention the inhibitor or the modulator is selected from the group consisting of an inhibitor of receptor interacting protein 1 (RIPK1), an inhibitor of receptor interacting protein 3 (RIPK3), an inhibitor of mixed lineage kinase domain like (MLKL) or a combination thereof.

In a further preferred embodiment of the inhibitor of the invention or the methods of the invention the inhibitor or the modulator is an inhibitor of RIPK3.

In yet another preferred embodiment of the inhibitor of the invention or the methods of the invention the inhibitor or the modulator is selected from the group consisting of necrostatin-1 (Nec-1; 5-(1H-indol-3-ylmethyl)-3-methyl-2-thioxo-4-omidazolidinone, 5-(indol-3-ylmethyl)-3-methyl-2-thio-hydantoin), necrostatin-1 stable (54(7-chloro-1H-indo1-3-yl)methyl)-3-methyl-2,4-imidazolidinedione, 54(7-chloro-1H-indo1-3-yl)methyl)-3-methylimidazolidine-2,4-dione), necrostatin-1 inactive (5-((1H-indo1-3-yl)methyl)-2-thioxoimidazolid in-4-one), necrosulfonamide (NSA; (E)-N-(4-(N-(3-Methoxypyrazin-2-yl)sulfamoyl)phenyI)-3-(5-nitrothiophene-2-yl)acrylamide), an anti-RIPK3 siRNA, an anti-MLKL siRNA or a combination thereof.

In another preferred embodiment of the method of modulating the transmigration of metastasising tumour cells through endothelium, the modulator is an inhibitor of TGF-beta-activating kinase 1 (TAK1).

As used herein the term “TAK1” refers to TGF-beta activated kinase 1 (also known as mitogen-activated protein kinase kinase kinase 7 (MAP3K7) a member of the serine/threonine protein kinase family which controls a variety of cell functions including transcription regulation and apoptosis. Further, it was shown that absence or inhibition of TAK1 results in increased necroptosis in endothelial cells (Morioka et al., 2012). Importantly, as shown in Example 4 herein below endothelial cells in which TAK1 was silenced using siRNA showed increased necroptosis upon stimulation with tumour cells. Furthermore, mice that lack TAK1 specifically in endothelial cells also showed increased necroptotic cell death in endothelial cells which coincided with increased tumour metastasis. Animals in which the necroptosis-related molecule RIPK3 was additionally deleted showed reduced metastasis formation. Thus, an inhibitor of TAK1 can be used to increase necroptosis. An inhibitor of TAK1 can be chosen from any of the classes herein above. Preferably, the TAK1 inhibitor inhibits expression of TAK1. For example, the TAK1 inhibitor may be an siRNA or shRNA specific for TAK1. An inhibitor of TAK1 is, thus, an activator/enhancer of necroptosis in accordance with the invention.

In a further preferred embodiment of the inhibitor of the invention or the method of the invention, the inhibitor or modulator is an inhibitor of death receptor 6 (DR6).

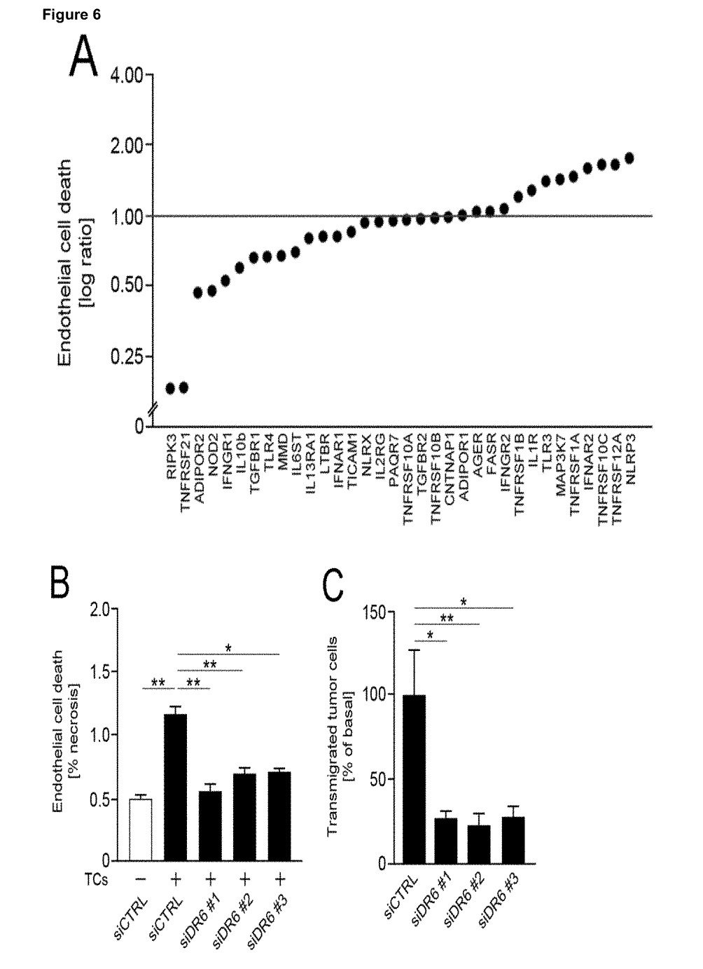

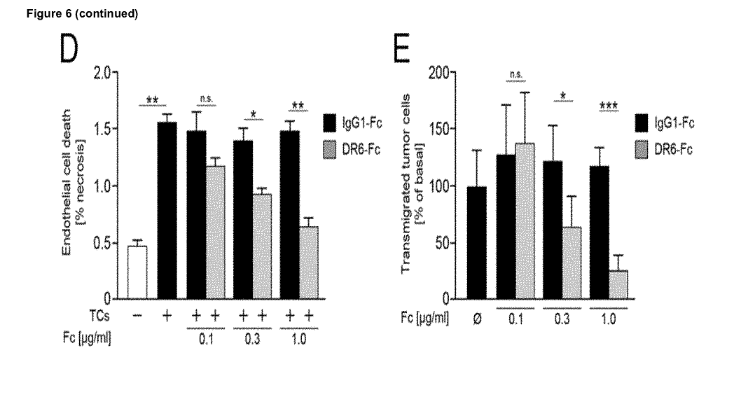

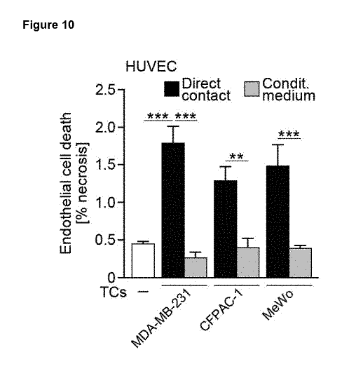

The term “DR6” as used herein refers to death receptor 6, also known as tumour necrosis factor receptor superfamily member 21 (TNFRSF21), a member of the TNFR superfamily. DR6 was considered to be an orphan receptor but recently it was found in neurons that the N-terminus of amyloid-precursor protein (N-APP) is a ligand of DR6 that induces cell death in neurons (Nikolaev et al., 2009, Nature, 457(7232):981-9). As shown in FIG. 6 herein below, siRNAs specific for DR6 or a DR6-Fc fusion protein decrease endothelial necroptotic cell death induced by tumour cells and transmigration of tumour cells, respectively. Thus, an inhibitor of DR6 may be employed to prevent necroptosis in endothelial cells and transmigration of metastasising tumour cells. An inhibitor of DR6 can be chosen from any of the inhibitor classes described herein above. In particular, a DR6 inhibitor may prevent the expression of the receptor, may interfere with its shuttling to the plasma membrane or may prevent its binding to its ligand (for example via decreasing the number of available ligands; this can, e.g., be achieved via siRNA against the ligand APP in tumor cells; see FIGS. 10 to 12) or may interfere with the stimulation-dependent binding to intracellular components and thus interfere with the activation of further downstream targets resulting in an activation of cell death. Preferably, the DR6 inhibitor is a siRNA specific for DR6 or a protein preventing binding of the ligand to DR6. For example, a decoy receptor, an antagonistic antibody against DR6 or a soluble form of the receptor, such as a fusion protein comprising the extracellular part of the receptor and the Fc-part of an antibody (DR6-Fc protein) may be used as an inhibitor (see also FIGS. 6D and 6E, 8 and 9). SiRNAs specific for DR6 are known in the art and can be obtained, for example, from SIGMA or Qiagen. DR6-Fc fusion proteins are also known in the art and are available commercially, for example from R&D Systems.

Further, in another preferred embodiment of the inhibitor of the invention the inhibitor for use in preventing the metastasis of tumours has admixed thereto or is associated in a separate container with a further pharmaceutically active agent.

Envisaged are, for example, combinations of anti-tumour agents with the inhibitors according to the invention. Anti-tumour agents are used in chemotherapy, are known in the art and include, e.g., antineoplastic drugs that kill tumour cells or inhibit their proliferation. Anti-tumour agents can be classified by their method of action or origin and include, for example, alkylating agents, anti-metabolites, plant alkaloids and terpenoids, topoisomerase inhibitors and cytotoxic antibodies. Exemplary alkylating agents include, e.g., nitrogen mustards (mechlorethamine, cyclophosphamide, ifosfamide, melphalan, chlorambucil), methylhydrazine derivative (Procarbazine (N-methylhydrazine, MIH)), alkyl sulfonate (busulfan), nitrosoureas (carmustine (BCNU), streptozocin (streptozotocin), bendamustine), triazenes (dacarbazine (DTIC, dimethyltriazenoimidazole carboxamide), temozolomide) platinum coordination complexes (cisplatin, carboplatin, oxaliplatin). Exemplary antimetabolites include, e.g., folic acid analogs (methotrexate (amethopterin), pemetrexed), pyrimidine analogs (fluorouracil (5-fluorouracil; 5-FU), capecitabine, cytarabine (cytosine arabinoside), gemcitabine, 5-aza-cytidine, deoxy-5-aza-cytidine), purine analogs and related inhibitors (mercaptopurine (6-mercaptopurine; 6-MP), pentostatin (2′-deoxycoformycin), fludarabine, clofarabine, nelarabine). Exemplary natural products (e.g., plant alkaloids and terpenoids) include, e.g., vinca alkaloids (vinoblastine, vinorelbine, vincristine), taxanes (paclitaxel, docetaxel), epipodophyllotoxins (etoposide, teniposide), camptothecins (topotecan, irinotecan), antibiotics (dactinomycin (actinomycin D), daunorubicin (daunomycin, rubidomycin), doxorubicin), echinocandins (yondelis), anthracenedione (mitoxantrone, bleomycin, mitomycin C), enzymes (L-asparaginase).

Exemplary hormones and antagonists include, e.g., adrenocortical suppressants (mitotane (o.p′-DDD)), adrenocortico-steroids (prednisone (other equivalent preparations available)), progestins (hydroxyprogesterone caproate, medroxyprogesterone acetate, megestol acetate), estrogens (diethylstilbestrol, ethinyl, estradiol (other preparations available), anti-estrogens (tamoxifen, toremifene), aromatase inhibitors (anastrozole, letrozole, exemestane), androgens (testosterone propionate, fluoxymesterone (other preparations available)), anti-androgen (flutamide, casodex), GnRH analog (leuprolide). Exemplary further anti-tumour agents include, e.g., substituted urea (hydroxyurea), differentiating agents (tretinoin, arsenic trioxide, histone deacetylase, inhibitor (vorinostat)), protein tyrosine kinase inhibitors (imatinib, dasatinib, nilotinib, gefitinib, erlotinib, sorafenib, sunitinib, lapatinib), proteasome inhibitor (bortezomib), biological response modifiers (interferon-alfa, interleukin-2), immunomodulators (thalidomide, lenalidomide), mTOR inhibitors (temsirolimus, everolimus) and monoclonal antibodies.

Also envisaged is the combination of inhibitors according to the invention with other metastasis preventing agents. Examples of such combinations can be found in G. Y. Perret, M. Crepin, Fundam. Clin. Pharmacol. 22, 465-492 (2008). A suitable treatment regimen of the combination of active agent and inhibitor as defined herein in the case of the latter being admixed or the further pharmaceutically active agent being provided separately in a container can be determined by routine methods. A container may take the form of, e.g., a vial. The vial may, in addition to the pharmaceutically active agent, comprise preservatives or buffers for storage, media for maintenance and storage. The pharmaceutically active agent may further be comprised in a pharmaceutical composition. The definitions given herein above with regard to the term “pharmaceutical composition” apply mutatis mutandis also to this embodiment.

It will be appreciated that this embodiment is suitable to provide a more complete clinical approach in disease management of subjects afflicted with tumours as defined herein above, i.e. known to metastasise or speculated to metastasise.

Further, the invention relates to a method of identifying necroptotic and necrotic cells comprising the steps of: a) contacting cells with a marker for plasma membrane breakdown; b) contacting said cells with a marker for chromatin condensation and/or chromatin fragmentation; and c) determining whether plasma membrane breakdown and chromatin condensation and/or chromatin fragmentation occurs, wherein it is indicative that said cells are necroptotic or necrotic, if plasma membrane breakdown is determined in step c) in the absence of chromatin condensation and/or chromatin fragmentation as occurring in apoptotic cells.

The term “necroptotic and necrotic cells” refers to cells which have died or are dying by necroptosis and necrosis, respectively.

In particular, in accordance with the method of the invention, necroptotic/necrotic cells are distinguished from living cells on the one hand and from cells which have died or are dying by another type of death, in particular from apoptotic cells, on the other hand.

As used herein the term “contacting cells with a marker for plasma membrane breakdown” refers to bringing the cells and the marker, i.e. a substance suitable for indicating permeabilisation of the plasma membrane, into such close proximity and under such conditions that an interaction between the marker and the cells can take place. In particular, the conditions are such that the marker can enter into the cell, if the plasma membrane is permeabilised, but is excluded from the cells, if the plasma membrane is intact. This requires that the conditions do not cause permeabilisation of the plasma membrane. Conditions that influence the integrity of the plasma membrane are well known in the art and include for example temperature, O₂-concentration, pH, salt content of the medium and the presence of a detergent.

As used herein the term “marker for plasma membrane breakdown” refers to a substance indicating that the plasma membrane of a cell is no longer fully intact, i.e. that the passage of compounds from the outside of the cell to its inside is no longer efficiently regulated. For example, the marker may be a substance which cannot cross the plasma membrane as long as the membrane is intact, i.e. a membrane-impermeable substance. Preferably, the marker is designed in a manner that allows for its convenient detection so that the location of the marker, i.e. whether the marker is located inside our outside a cell, can be easily determined. For example, the marker may be a dye in itself or it may be labelled. Suitable labels are well known in the art. Preferably, the marker is a fluorescent dye or has a fluorescent label. It is further preferred that the marker for plasma membrane breakdown is a membrane-impermeable DNA-binding fluorescent dye.

Most preferably, the marker for plasma membrane breakdown is Ethidium homodimer III (EtDIII).

The term “marker for chromatin condensation and/or chromatin fragmentation” refers to a substance which allows for the distinction between different chromatin states. For example, the marker may either stain chromatin as such in which case the pattern of the stained area indicates whether chromatin is condensed and/or fragmented or the marker may bind only to one of the states of chromatin, e.g. only to the condensed and/or fragmented state. In the latter case, the presence of a staining is indicative of the chromatin being condensed and/or fragmented.

In order to be able to stain chromatin, the substance needs to be able to get into the cell and in particular into the nucleus. Thus, the marker for chromatin condensation and/or chromatin fragmentation preferably is a membrane-permeable substance. For easier detection it is preferred that the marker for chromatin condensation and/or chromatin fragmentation is either a dye in itself or is labelled. Preferably the marker is fluorescent or has a fluorescent label. It is further preferred that the marker for chromatin condensation and/or chromatin fragmentation is a membrane-permeable DNA-binding dye. It is further preferred that the marker is suitable for staining cells in their natural state, i.e. that it can be used on non-fixed cells. Most preferably the marker for chromatin condensation and/or chromatin fragmentation is Hoechst 33342.

It is further preferred that the marker for chromatin condensation and/or chromatin fragmentation can be distinguished from the marker for plasma membrane breakdown upon detection. For example, the two markers may emit fluorescence at a different wavelength, thus allowing for their distinction.

The step of contacting the cells with a marker for plasma membrane breakdown (step a)) may be performed before, after or simultaneously with contacting said cells with a marker for chromatin condensation and/or chromatin fragmentation (step b)). The step of determining whether plasma membrane breakdown has occurred (step c)) is performed after the marker for plasma membrane breakdown has been added, i.e. after step a) and the step of determining whether chromatin condensation and/or chromatin fragmentation have occurred (step c)) is performed after the cells have been contacted with the marker for chromatin condensation and/or chromatin fragmentation (step b)). Preferably, step c) is performed after steps a) and b). If plasma membrane breakdown has occurred, i.e. if a positive staining with the marker for plasma membrane breakdown is observed, and if, at the same time, no chromatin condensation and/or chromatin fragmentation as occurring in apoptotic cells has occurred, i.e. if there is no staining indicative for chromatin fragmentation and/or a staining indicative for chromatin condensation is absent or weak as compared to such a staining in apoptotic cells, the cell is considered to be necroptotic/necrotic (see Example 1, section “Cell death analysis” for further details, which can be referred to in general). Also preferred is, that the step (c) is performed by light microscopy.

The loss of plasma membrane integrity, i.e. a positive staining with the marker for plasma membrane breakdown, is indicative of the cell being dead or being in the process of dying. However, such a staining does not allow for late apoptotic and necroptotic/necrotic cells to be distinguished. On the other hand, apoptosis is often associated with chromatin fragmentation, whereas this feature is absent in necroptotic/necrotic cells. Further, the degree of chromatin condensation during necroptosis/necrosis, if any, is very moderate. Usually, during necroptosis/necrosis there is a modest “shrinkage” of the nucleus which can also be interpreted as weak condensation, while during apoptosis there is a real (much stronger) condensation. This condensation is a marker for apoptosis also in the absence of additional fragmentation. Therefore, the combination of plasma membrane breakdown, presence or absence of chromatin fragmentation and the degree of chromatin condensation allows the discrimination between apoptotic and necroptotic/necrotic cell death. The presence of plasma membrane breakdown in the absence of chromatin fragmentation and/or chromatin condensation as occurring in apoptotic cells is indicative of a necroptotic cell.

As used herein, the term “in the absence of chromatin condensation and/or chromatin fragmentation as occurring in apoptosis” indicates that no chromatin fragmentation is present and that the chromatin condensation, if any, is weak as compared to the chromatin condensation observed in an apoptotic cell. Preferably, no chromatin fragmentation is present and (i) there is no chromatin condensation or (ii) the chromatin condensation that is present is markedly reduced as compared to the chromatin condensation in an apoptotic cell. It is understood that the chromatin condensation in an apoptotic cell which is used as a reference does not have to be the chromatin condensation in a specific apoptotic cell. Instead, the average amount of chromatin condensation generally observed in apoptotic cells may be used as a reference. Generally, it is known in the art how to determine whether the degree of chromatin condensation or fragmentation in a given cell is typical of an apoptotic cell. In particular in combination with a plasma membrane breakdown marker, the cells can be classified without further ado on the basis of knowledge in the art.

The method can be scaled up for the analysis of a large amount of samples, for example, by performing the assay in a 96-well, 384-well or 1536-well format and by using automated image acquisition and semi-automated image analysis of the respective morphological criteria.

To further corroborate the distinction between necrotic/necroptotic and apoptotic cells it is possible but not required to combine the method with other methods of detecting apoptotic cells such as for example TUNEL assay or staining of cleaved caspase 3.

So far, the most reliable method to discriminate apoptotic from necroptotic cell death is based on the analysis of morphological features by electron microscopy. However, this method is extremely laborious and not feasible for the analysis of large amounts of samples or to study the systemic relevance in an organism. Other methods often are unable to distinguish between late apoptotic cells and necroptotic cells. In contrast, the method of the present invention provides a method which allows the discrimination between necrotic/necroptotic versus apoptotic cells, which is less laborious and facilitates the analysis of large amounts of samples (see Example 2). Moreover, the method of the present invention can also be applied in vivo as shown in Examples 4 and 5 herein below. In addition, it does not rely on the indirect analysis of the type of cell death by specifically blocking one type of cell death. Thus, the method of the present invention, contrary to indirect methods of the prior art, is not associated with the risk of artificially altering the type of cell death. Furthermore, in accordance with the method of the present invention, cells can be analysed individually, thus allowing for high sensitivity of the analysis. Based on the method of the present invention a very precise distinction between apoptotic and necroptotic cell death can be achieved. In addition, even subtypes of the respective cell death form (i.e. early and late apoptosis vs. early and late necroptosis) can be visualised and quantified. By contrast, known methods often fail to discriminate late apoptotic cells from necroptotic cells (where in both cases a loss of plasma membrane integrity occurs).

In a preferred embodiment of the method of identifying necroptotic and necrotic cells the marker for plasma membrane breakdown in step a) is selected from the group consisting of a membrane-impermeable DNA-binding dye and/or the marker for chromatin condensation and/or chromatin fragmentation is selected from the group consisting of membrane-permeable DNA-binding dyes.

Using a membrane-impermeable DNA-binding dye as a marker for plasma membrane breakdown is advantageous as compared to using other membrane-impermeable markers because dyes that stain for example organelles are more difficult to detect since labelled organelles may be too small to be visualised with non-confocal microscopy. Thus, methods based on such dyes may require more complicated detection systems and may be more laborious. A membrane-impermeable dye staining the cytoplasm might result in a strong background as the dye might simply leak out. These disadvantages are avoided by using a membrane-impermeable DNA-binding dye as a marker for plasma membrane breakdown. Further, a non-fluorescent, non-permeable dye that becomes fluorescent upon enzymatic activation is not suitable for staining necroptotic/necrotic cells because enzymatic activity is expected to be low or absent in necroptotic cells.

As regards the embodiments characterised in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all attached claims.

The figures show:

FIG. 1: Schematic overview on interactions of tumour, immune and endothelial cells during metastasis (adopted from Labelle and Hynes, 2012).

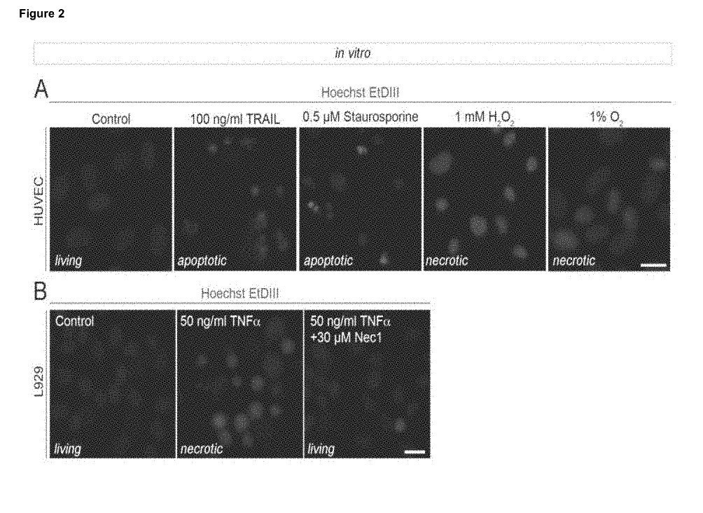

FIG. 2: Method to discriminate apoptotic from necrotic cell death A: Human Umbilical Vein Endothelial Cells (HUVEC) were treated with the indicated stimuli and analysed by staining with Hoechst 33342 and EtDlll. TRAIL and Staurosporine induced apoptosis whereas cells treated with H₂O₂ or O₂ showed a necrotic phenotype. B: TNF treatment resulted in a necrotic phenotype in L929 cells which could be blocked by addition of necrostatin-1 as shown by staining with Hoechst 33342 and EtDlll.

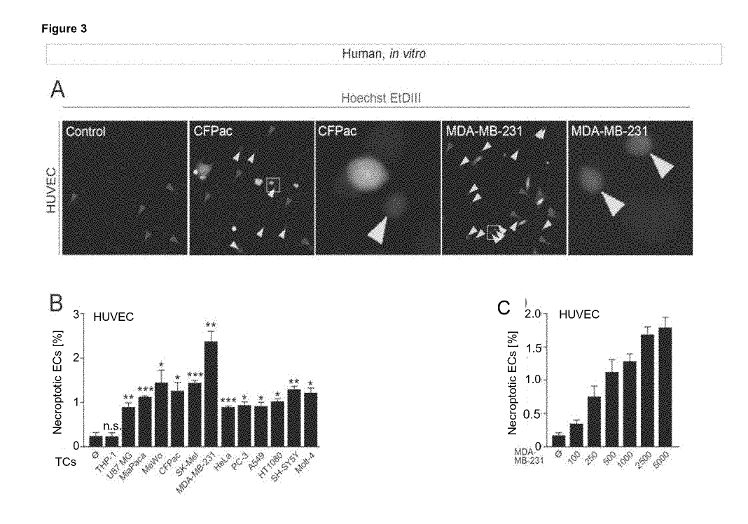

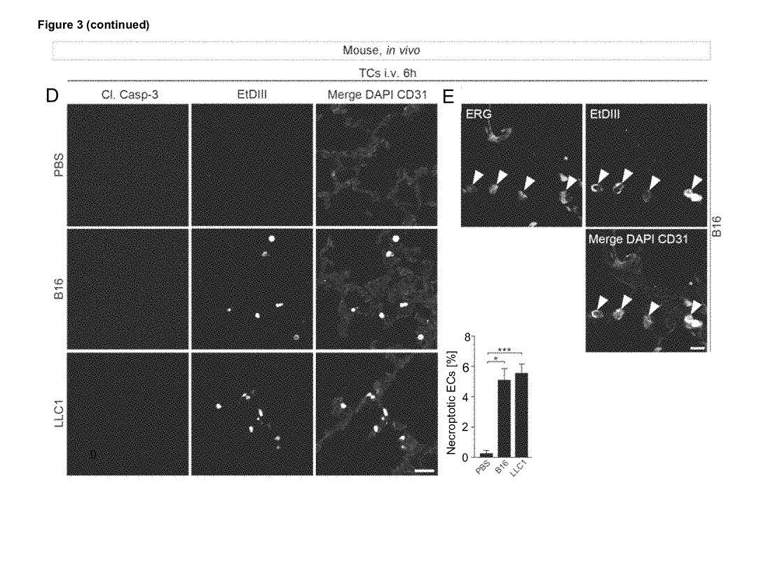

FIG. 3: Tumour cells (TCs) induce necroptosis in endothelial cells (ECs) in vitro A: CFPac and MDA-MB-231 tumour cells induce endothelial cell necroptotic cell death in HUVEC in vitro as shown by staining with Hoechst 33342 and EtDlll. B: Quantification of necroptotic HUVEC after treatment with different TCs. C: Quantification of necroptotic HUVEC after treatment with increasing numbers of MDA-MB-231 TCs. D: Intravenously injected TCs (B16 or LLC1) induce death of endothelial cells of the mouse lung in vivo. This death is necroptotic as shown by positive staining with EtDlll in the absence of nuclear condensation or fragmentation as shown by DAPI staining. Further, cleaved caspase could not be detected. E: CD31 and ERG served as endothelial specific markers.

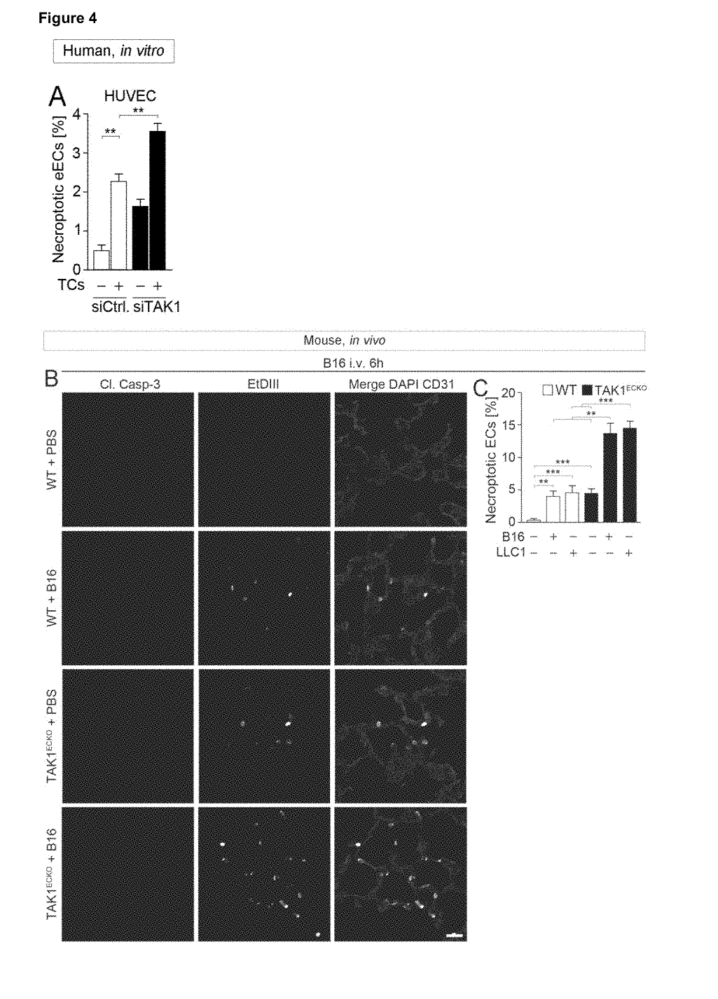

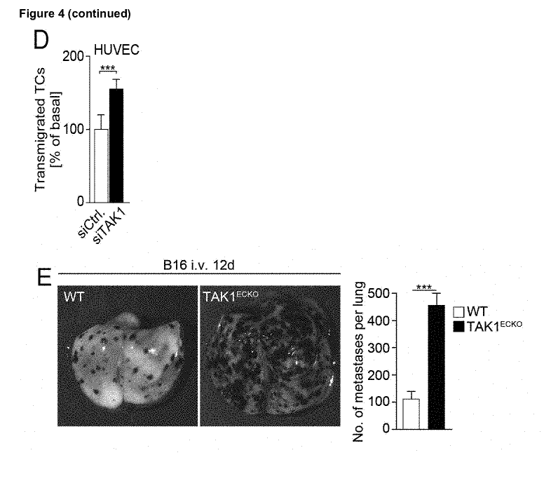

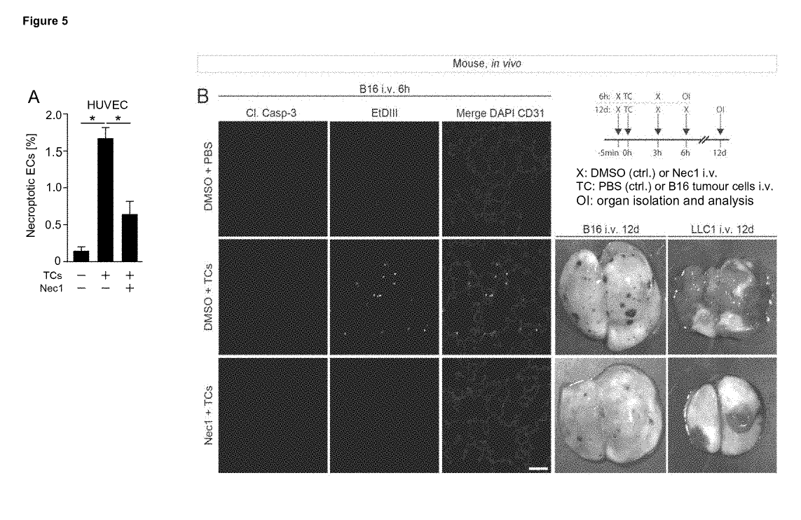

FIG. 4: Increased necroptosis in endothelial cells (ECs) leads to increased tumour cell (TC)-induced endothelial cell death and metastasis. A: Deletion of TAK1 increases TC-induced necroptosis in HUVEC. B: Necroptosis induced by TCs (B16) in endothelial cells in vivo is increased in mice lacking TAK1 in endothelial cells)(TAK1^{ECKO} as compared to wild type (wt) mice. C: Quantification of necroptotic ECs induced by TCs (B16 or LLC1) in wt and TAK1^{ECKO} mice D: Silencing of TAK1 increases the number of transmigrated TCs. E: Increased metastasis formation by TCs (B16) in TAK1^{ECKO} mice as compared to wt mice.

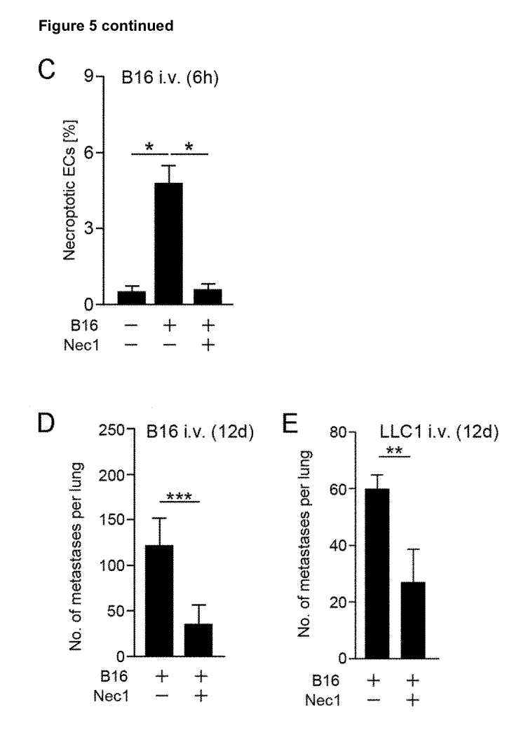

FIG. 5: Pharmacological inhibition of tumour cell (TC)-induced endothelial cell (EC) death results in reduced metastasis formation. A: Necrostatin 1(Nec-1) reduces the number of TC-induced necroptotic HUVEC in vitro. B: Nec-1 reduces TC (B16) induced necroptotic ECs and metastasis formation by TCs (B16 or LLC1) in vivo. C: Quantification of the effect of Nec-1 on the number of TC (B16)-induced necroptotic ECs in vivo. D: Quantification of B16 metastasis in the presence and absence of Nec-1 in vivo. E: Quantification of LLC1 metastasis in the presence and absence of Nec-1 in vivo.

FIG. 6: DR6 is required for tumour cell (TC) induced endothelial cell (EC) death and TC transmigration A: siRNA screen to identify potential receptors that mediate TC-induced necroptotic EC death (RIPK3 and MAP3K7 (=TAK1) served as internal controls) B: confirmation of the screening hit (DR6) with different siRNAs C: reduced transendothelial migration of TC through a HUVEC monolayer with silenced DR6 expression (knockdown efficiency >70%, data not shown) D: effect of DR6-Fc fusion proteins (R&D Systems, #144-DR) on TC-induced necroptotic EC death E: effect of DR6-Fc fusion proteins on TC transendothelial migration through a HUVEC monolayer.

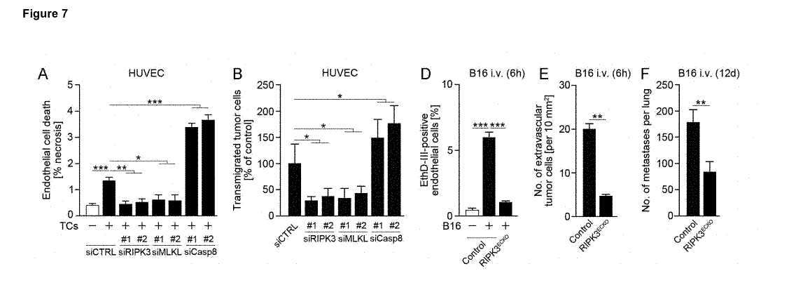

FIG. 7: Tumor cell-induced endothelial necroptosis and metastasis formation are regulated by endothelial RIPK3 and raspase-8 (A and B) Endothelial cells (HUVECs) were transfected with control siRNA (siCTRL) or with siRNAs directed against the messenger RNAs encoding RIPK3 (siRIPK3), MLKL (siMLKL) or caspase-8 (siCasp8), and (A) endothelial cell death was determined in the absence of tumor cells (−TC, white bar) or presence of MDA-MB-231 tumor cells (+TC, black bars). The number of transmigrated MDA-MB-231 tumor cells over a HUVEC monolayer after siRNA-mediated gene knockdowns as indicated was determined in (B) (n=6 wells per condition). (C) Effects of endothelial-specific deletion of RIPK3(RIPK3^{ECKO}) on tumor cell-induced endothelial cell death (6 h, left panel) and metastasis formation in vivo (12d, right panel).

Schematic on the top right shows the experimental design. 6 h: representative confocal images of lung sections 6 h after i.v. injection of B16 tumor cells into animals of the indicated genotype and stained for cleaved caspase 3 (providing a green colour), EthD-III (providing a yellow colour), CD31 (providing a red colour) and cell nuclei (DAPI, providing a blue colour). 12d: representative images of lungs 12d after i.v. injection of B16 tumor cells into animals of the respective genotype. Cre-negative littermates served as control. Bar length: 50 μm. (D-F) Quantification of (D) EthD-III-positive endothelial cells at 6 h, (E) number of extravasated tumor cells at 6 h or (F) lung metastases at 12d after i.v. injection of B16 tumor cells into RIPK3^{EcKQ} mice. Quantifications in (D) and (F) are based on images as shown in (C). The white bar in (D) represents mice that received PBS instead of tumor cells (n=4-6 animals per condition). All panels are representative results of three or more independent experiments. Shown are mean values ±SEM (A, D, E) or ±SD (B, F); *p<0.05; **p <0.01; ***p<0.001 (one-way ANOVA and Bonferroni's post hoc test).

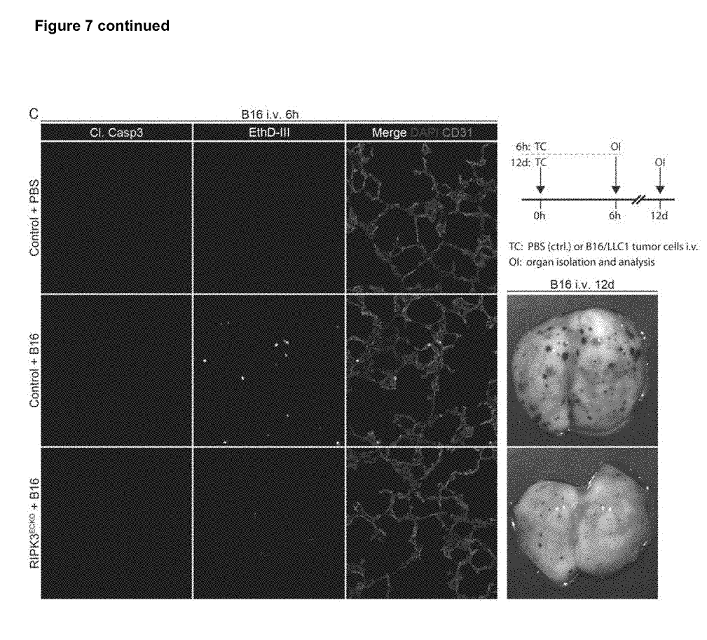

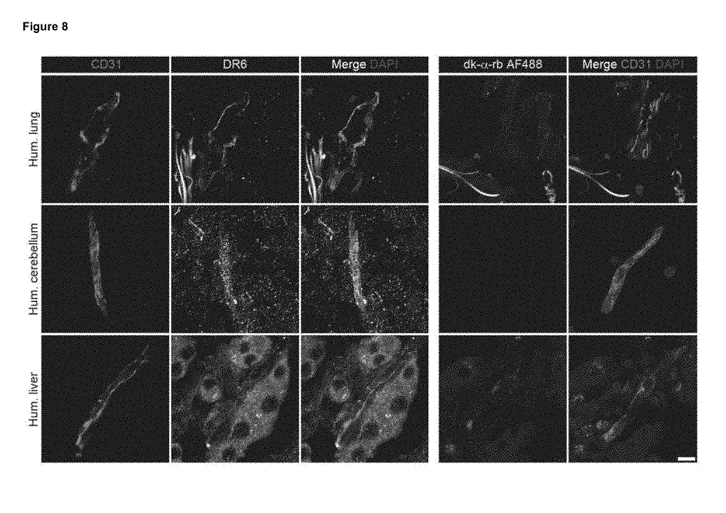

FIG. 8: DR6 is expressed in endothelial cells of various human organs Representative confocal images of human tissues of the indicated origin stained for CD31 (providing a red colour), DR6 (providing a green colour) and cell nuclei (DAPI, providing a blue colour) (left panel). HUVECs in which DR6 was knocked-down (siDR6) served as control for antibody-specificity (lower panel). No perrneabilization agents were used for the stainings. Control-IgG antibody and donkey anti-rabbit secondary antibody coupled to AF488 served as negative controls (right panels). Bar length: 5 μm.

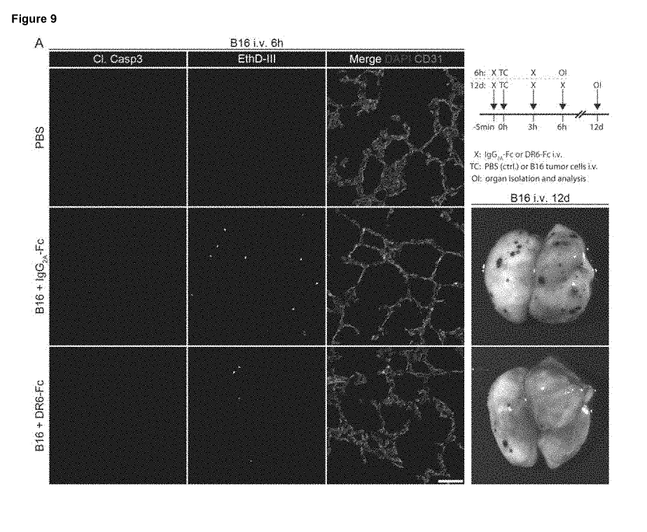

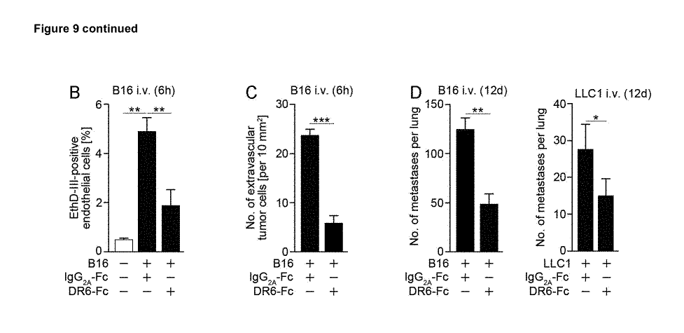

FIG. 9: Ligand binding to DR6 induces endothelial necroptosis and promotes metastasis formation (A) Effects of (mouse) IgG₂ -Fc or DR6-Fc fusion proteins (0.2 pgig per dose) on tumor cell-induced endothelial cell death (6 h, left panel) and metastasis formation in vivo (12d, right panel). Schematic on the top right shows the experimental design. 6 h: representative confocal images of lung sections 6 h after i.v. injection of B16 tumor cells and treatment as indicated. Control animals received PBS instead of tumor cells. Tissue sections were stained for cleaved caspase 3 (green), EthD-III (yellow), CD31 (red) and cell nuclei (DAPI, blue). 12d: representative images of lungs 12d after i.v. injection of B16 tumor cells and the respective treatment. Bar length: 50 μm. (B-D) Quantification of (B) EthD-III-positive endothelial cells at 6 h, (C) number of extravasated tumor cells at 6 h or (D) lung metastases at 12d after i.v. injection of B16 or LLC1 tumor cells into mice treated as indicated. Quantifications in (B) and (D) are based on images as shown in (A). The white bar in (B) represents mice that received PBS instead of tumor cells (n=4-6 animals per condition).

All panels are representative results of three or more independent experiments. Shown are mean values ±SEM (B, C) or ±SD (D); *p<0.05; **p<0.01; ***p<0.001; n.s., not significant (one-way (B) ANOVA and Bonferroni's post hoc test or unpaired, two-tailed Student's t-test (C, D)).

FIG. 10: Direct tumor cell-endothelial cell contact is required for the induction of endothelial necroptosis Quantification of cell death (necrosis) in HUVECs alone (white bars) or cultured in the presence of different types of tumor cells as indicated (black bars, direct contact) or stimulated with conditioned medium from overnight co-cultures from endothelial cells with tumor cells (grey bars, condit. medium) Shown are mean values ±SEM; **p<0.01; ***p<0.001; n.s. not significant (one-way ANOVA and Bonferroni's post hoc test).

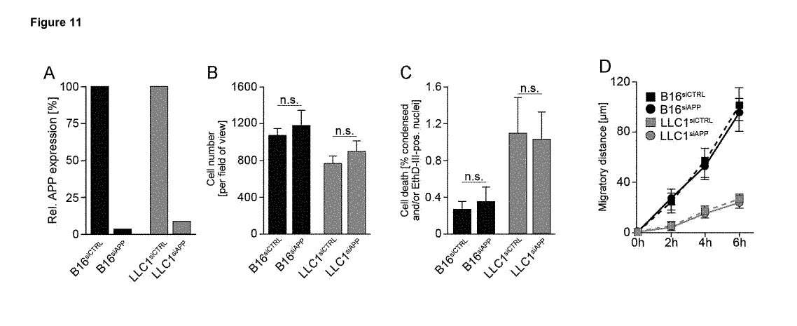

FIG. 11: APP-deficiency in tumor cells has no effect on tumor cell proliferation, cell death or migration (A) Analysis of knockdown efficiency in B16 or LLC1 tumor cells using siRNAs against APP (siAPP). Scrambled siRNA (siCTRL) served as control. Shown is the relative mRNA expression normalized to GAPDH levels and to the level detected in scramble siRNA-treated samples. (B-D) Quantitative evaluation of (B) cell numbers, (C) cell death or (D) the migratory ability of B16 or 1.1C1 tumor cells transfected with siRNA against APP (siAPP) as compared to tumor cells transfected with scramble siRNA (siCTRL) (n=3-6 wells per condition).

All panels are representative results of three or more independent experiments. Shown are mean values ±SD; ***p<0.001; n.s., not significant (unpaired, two-tailed Student's t-test).

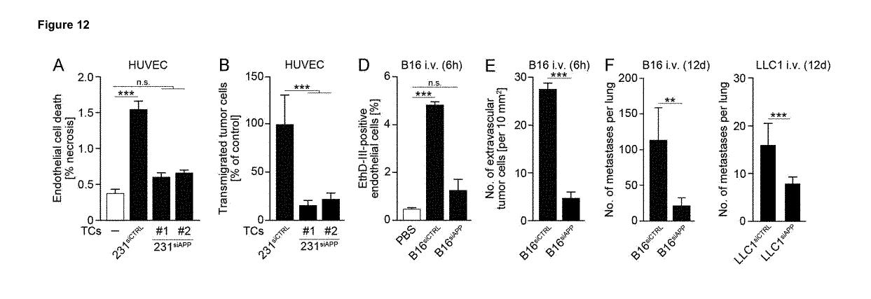

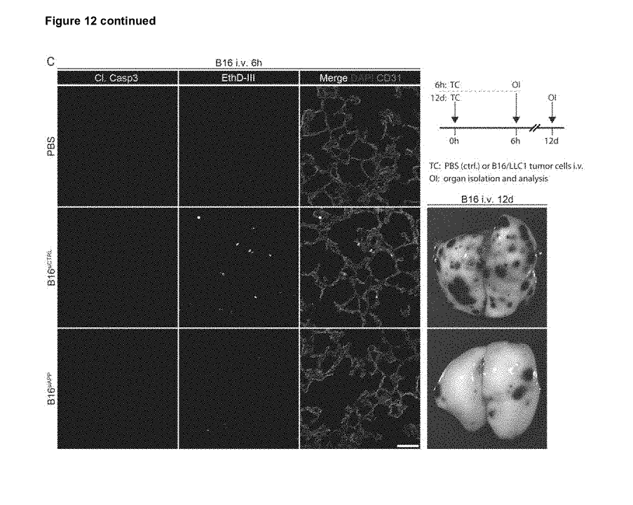

FIG. 12: APP expressed by tumor cells induces endothelial necroptosis and promotes metastases formation (A and B) Quantification of (A) cell death (necrosis) in HUVECs in the absence of tumor cells (-TC, white bar) or exposed to MDA-MB-231 tumor cells transfected with siRNA against mRNA encoding APP (₂₃₁ sip,: or control siRNA (231^{scIRL}) (+TC, black bars) Transendothelial migration of 231^{siCTRL} or 231^{siAPP} tumor cells over a HUVEC monolayer was determined in (B) (n=6 wells per condition). (C) Effects of B16 tumor cells with silenced APP expression (B16^{siAPP}) on endothelial cell death (6 h, left panel) and metastasis formation in vivo (12d, right panel). Schematic on the top right shows the experimental design. 6 h: representative confocal images of lung sections 6 h after i.v. injection of B16^{siAPP} or B16^{siCTRL} tumor cells and stained for cleaved caspase 3 (providing a green colour), EthD-III (providing a yellow colour), CD31 (providing a red colour) and cell nuclei (DAPI, providing a blue colour). 12d: representative images of lungs 12d after i.v. injection of the respective tumor cells. Bar length: 50 μm. (D-F) Quantification of (D) EthD-III-positive endothelial cells at 6 h, (E) number of extravasated tumor cells at 6 h or (F) lung metastases at 12d after i.v. injection of APP-silenced (B16^{siAPP} and LLC1^{siAPP}) or control B16 tumor cells (B16^{siCTRL}) and control LLC1 tumor cells (LLC1^{CTRL}). Quantifications in (D) and (F) are based on images as shown in (C). The white bar in (D) represents mice that received PBS instead of tumor cells (n=4-6 animals per condition).

Shown are mean values ±SEM (A, D, E) or ±SD (B, F); **p<0.01; ***p<0.001; n.s., not significant (one-way ANOVA and Bonferroni's post hoc test (A, B, D) or unpaired, two-tailed Student's t-test (E, F)).

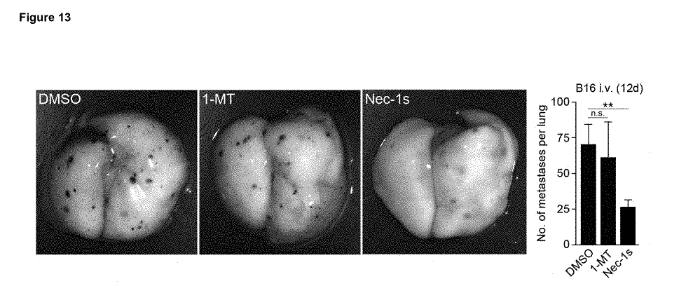

FIG. 13:

Representative images and quantification of metastases formed in the lungs of animals upon i.v. injection of DMSO, 1-methyltryptophan (1-MT, 1.65 μg/g per dose) or the stable isoform of Nec-1 (Nec-ls, 1.65 μg/g per dose) shortly before and at 3 h and 6h after B16 tumor cell injection into the tail vein. Formation of lung metastases was determined 12d thereafter (n=4-5 animals per condition).

All panels are representative results of three or more independent experiments. Shown are mean values ±SD; **p<0.01; n.s., not significant (one-way ANOVA and Bonferroni's post hoc test).

The examples illustrate the invention:

Example 1: Methods

In vitro:

Endothelial cells were grown to reach 80-90% confluency before addition of Ethidium homodimer III (EtDIII) alone or together with substances or with tumour cells together with substances or inhibitors as indicated in the figures. After 6-18 h, cells were stained with Hoechst33342 and images were acquired. In order to identify the mode of cell death, all images were analyzed with FIJI. The total number of nuclei was determined through a low threshold over all Hoechst positive nuclei. On the same channel a second, separate threshold was used to determine the number of condensed nuclei. A third threshold on the second channel was used in order to determine the number of nuclei that were stained positive for EtDlll. Knockdown in cells was achieved by double-transfection with siRNA using Lipofectamie RNiMAX.

In vivo: Tumour cells and substances or inhibitors were injected i.v. as depicted in the scheme in FIG. 5B into wildtype animals or animals of the respective genotype. To identify EtDIII-positive cells in vivo, animals were injected with EtDIII i.v. 10 min before sacrifice. Organs were isolated and processed for immunohistochemical analysis. For determining the number of metastasis, animals were sacrificed, lungs were isolated and macroscopically analyzed. All reagents used for these studies (incl. DR6-Fc) are commercially available (e.g. Lonza, CellSignaling, Sigma, R&D, etc.).

Cell Death Assays: HUVECs, HMVECs-L or L929 cells (1.5×10⁴ at seeding in 100 μl) were cultured for 24 hours in 96-well plates. To induce cell death, cells were stimulated overnight with either rhTRAIL (100 ng/ml, Peprotech), Staurosporine (0.5 pM, Jena BioScience), H₂O₂ (1 mM, AppliChem) or rmTNFa (100 ng/ml, Peprotech) or cultured under hypoxic conditions (1% O₂, Coy Laboratory Inc). Alternatively, for co-culture experiments, 1.5×10³ GFP-expressing tumor cells, Calcein-AM-labeled tumor cells, COS-1 or HEK cells (for more details see supplementary experimental procedues), or freshly isolated human peripheral blood mononuclear cells (PBMC) stained with Calcein-AM containing 20 times the number of platelets were added alone, in combination with each other or in the presence of the indicated substances onto the endothelial cell monolayer and cultured overnight: Nec-1 (30 μM), z-VAD-fmk (100 μM), 1-MT (30 μM), DR6-Fc or IgG1-Fc (0.1-1 μg/ml). PBMCs were isolated using standard protocols with Ficoll density gradient centrifugation. For supernatant experiments, HUVEC monolayers grown to confluency were cultured with conditioned medium obtained from HUVECs co-cultured in the presence of tumor cells for 18 h. For knockdown experiments, 1.5×10⁴ HUVECs were transfected using Lipofectamine RNAiMAX (Life Technologies) with different sets of siRNA (Sigma or Qiagen) and cultured on 96-well plates. Knockdown efficiencies were determined by Western Blotting using antibodies against RIPK3 (Abcam), Caspase-8 (ProSci) and a-tubulin (Sigma) upon lysis with Laemmli buffer or by quantitative PCR (Roche). In cases where siRNA-mediated knockdown was performed on tumor cells, cells were transfected using Lipofectamine RNAiMAX with different sets of siRNA (Sigma) and seeded 48h after transfection on confluent monolayers of HUVECs. Knockdown efficiencies were determined using quantitative PCR (Roche). Tumor cell number upon knockdown was determined by counting Hoechst33342-positive cells, and tumor cell death was determined by counting condensed and/or EthD-III-positive nuclei (see below). Cell migration was determined by a scratch assay Wang et al., 2007).

Cell Death Analysis: For all conditions, EthD-111 (1.6 μM, Biotium) was added to the medium before overnight culture, and Hoechst33342 (2 μM, Thermo Scientific) was added shortly before automated image acquisition in an atmosphere-controlled chamber (37° C., 5% CO2) using an Olympus IX81 microscope. Based on cells cultured under defined apoptotic, necrotic or necroptotic conditions and stained with Hoechst (a cellpermeable nuclear dye) and EthD-III (a membrane-impermeant nuclear dye), morphological criteria for discriminating apoptotic from necrotic (or necroptotic) cells as compared to living cells were defined as follows: a living cell appears with a normal round to kidney-shaped nucleus (as visualized by Hoechst) and is negative for EthD-III. An apoptotic cell appears with a strong condensed and frequently fragmented nucleus and is negative for EthD-III. A necrotic or necroptotic cell appears with a normal round to kidney-shaped nucleus or with a minor degree of nuclear shrinkage (no condensation and no fragmentation) and is positive for EthDIII.

A late apoptotic cell is positive for EthD-III but can be discriminated from a necrotidnecroptotic cell because of its strong condensation (and frequent fragmentation) of the nucleus. To all images to be analyzed, a Gaussian blur with a radius of 3 pixels was applied to prevent repeatedly counting of fragmented parts of apoptotic nuclei. Endothelial cells were defined as GFP- or Calcein-AM-negative cells. The total number of all endothelial nuclei was determined through a low threshold (TH1) and application of a watershed on the resulting binary image over all Hoechst positive nuclei (minus the nuclei from tumor cells). When possible, a second separate threshold (TH2) was used to determine the number of condensed nuclei.

The number of EthD-III-positive cells was determined through an independent second low threshold only. In cases where this automated analysis failed, the mode of cell death was determined manually for each individual endothelial cell by application of the criteria summarized above. All images were analyzed in lmageJ (NIH). Each experiment was performed at least three times with a minimum of six wells per condition and four independent images acquired per well.

Metastasis Models: 50 μl containing 5x10⁴ unlabeled or CFSE-labeled tumor cells (B16F10 melanoma or L.L.C1 lung carcinoma cells) or tumor cells with silenced APP expression or fluorescent microspheres (15 μm, Life Technologies) in PBS were injected to the lateral tail vein of mice. For inhibitory experiments, two doses of 50 μl Nec-1 (1.65 μg/g), Nec-1 s (1.65 μg/g), 1-MT (1.65 μg/g), z-VAD(OMe)-fmk (4 μg/g) or two doses of 25 μl of rmDR6-Fc or rmigG₂A.-Fc (each at 0.2 μg/g) were injected into the tail vein shortly before and 3 hours after tumor cell injection. In all cases, for evaluation of tumor cell-induced endothelial cell death, six hours after injection of tumor cells, 50 pi EthD-III (300 μM in PBS) were injected i.v. and after 10 minutes animals were sacrificed and perfused with PBS and 4% paraformaldehyde and directly processed for immunohistochemical analysis. For evaluation of the number of extravasated tumor cells, CFSE-labeled B16 tumor cells were injected i.v. and 6h later non-perfused lungs were isolated and fixed in 4% paraformaldehyde, Cryosections of tissues were stained for cleaved caspase 3 (CellSignal), Annexin V (Santa Cruz Biotechnology), ERG (Abcam) and CD31 or CD45 (BD Biosciences). DAPI (Life Technologies) was used to visualize nuclei.

TUNEL staining kit was from Roche. Sections were analyzed in XYZ views on a Leica SPS confocal microscope. The number of EthD-III-positive endothelial cells was determined by manual counting of EthD-III/ERG- or EthD-III/CD31-positive cells on a minimum of four random tissue sections per organ. For quantification of extravasating tumor cells, cryosections were analyzed by two criteria: tumor cells directly surrounded by CD31 staining (i.e. blood vessel) and with a noninvasive phenotype (i.e. round cell shape) were scored as intravascular, while cells outside blood vessels with an invasive phenotype (i.e. irregular cell shape with protrusions) were scored as extravascular. For evaluation of lung metastases, an additional (third) treatment with the aforementioned substances was performed at 6 hours after tumor cell injection, and lung metastases were analyzed macroscopically twelve days thereafter. A minimum of three animals per group was used. All experimental animal precedures were approved by the Hessian Regional Board.

Statistical Analysis: If not stated otherwise, one representative of at least 3 independently performed experiments is shown. In all studies, comparison of mean values was conducted with unpaired, two-tailed Student's f-test or one-way or two-way ANOVA with Bonferroni's post hoc test. In all analyses, statistical significance was determined at the 5% level (p<0.05). Depicted are mean values ±SD or ±SEM as indicated in the figure legends.

Materials: Media and supplements were from Life Technologies. Nec-1 was from Enzo Life Sciences. Nec-1s was from BioVision. Z-VAD-fmk was from Alexis and z-VAD(OMe)-fmk was from Cayman. 1-MT was from Sigma. rhDR6-Fc, rhlgG₁-Fc, rmDR6-Fc and rmIgG_{2A}-Fc were from R&D Systems. Calcein-AM was from AAT Bioquest, CFSE was from Alexis. Anti-DR6 antibody for Western Blot and immunohistochemical stainings was from Bioss.

Cells: Human primary endothelial cells and media were from Lonza. MDA-MB-231-GFP tumor cells were from AntiCancer. THP-1, A549, PC3, MeWo and SK-MEL-28 cells were from CLS. B16F10 and LLC1 were from ATCC. L929 cells were a kind gift from Jan Wiegers (Biocenter Innsbruck, Austria), U-87 MG cells were from Stefan Rieken (University Hospital, Heidelberg, Germany), MIA PaCa-2 and CFPAC-1 cells were from Nathalia Giese (University Hospital, Heidelberg, Germany), Sh-SY5Y, HeLa and HT1080 were from Michael Bahr (DKFZ, Germany) and MOLT-4 cells were from Jacek Witkowski (Medical

University of Gdansk, Poland). COS-1 cells were from ATCC. All cells were incubated at 37° C. and 5% CO₂. Human umbilical vein endothelial cells (HUVECs) and human microvascular vein endothelial cells from lung (1-IMVECs-L) were cultured in EGM2 or EGM2-MV medium, respectively, and passages <P6 were used for all experiments. All other cell lines were cultured in either RPMI or DMEM supplemented with 10% FBS, penicillinistreptomycin (100 unitsiml) and glutamine (2 mM). Primary mouse lung endotheliai cells were isolated and cultured as described previously (Sivaraj et al., 2013).

Transwell Assays: For a detailed description see (Schumacher et al., 2013). Briefly, for inhibitory experiments, HUVECs (1.5×10⁴ at seeding in 50 μl) were cultured for 2 days or, for knockdown experiments, 8×10³ HUVECs were transfected using Lipofectamine RNAIMAX with different sets of siRNA (Sigma or Qiagen) and cultured on 96-transwell plates with polyester membranes of 8-μm pore size (Corning) with daily medium changes until reaching confluency. For transmigration, the medium from the upper compartment was removed and 7.5×10³ GFP-expressing or Calcein-AMIabeled tumor cells were added in 50 μl EGM-2 medium alone or in the presence of different substances (see above). For all experiments, transmigrated tumor cells on the lower side of the filter were imaged (Zeiss Axio Observer.Z1 or Olympus IX81) and quantified with ImageJ. For permeability assays, EGM-2 medium containing 70 kDa FITC-Dextran (2 mg/ml) was added on top of the endothelial monolayer in the upper compartment. After 90 min, the amount of passed FITC-Dextran to the lower compartment containing EGM-2 only was measured (FlexStation3, Molecular Devices). Each experiment was performed at least three times with a minimum of five wells per condition.

Genetic Mouse Models: Control C57BI/6 animals were obtained from Charles River. To generate RIPK3 conditional knock-out animals, an 880-bp fragment containing loxPflanked exon 2 and 3 from ripk3 as well as the 5′ homology arm and the 3′ homology arm was amplified from BAC RPCI-23-237G18 (Children's Hospital Oakland Research Institute) and cloned into the pKOII targeting vector additionally containing a Frt-flanked neomycin resistance gene (neo^{R}) and the Diphtheria toxin A gene (dta) as negative selection marker. The targeting vector was linearized with Notl and introduced into V6.5 ES cells by electroporation. Upon treatment with 400 μg/ml G418, DNA from 400 clones was isolated, and screened for correct recombination by Southern Blot. Two independent ES cell clones were injected into C5761/6 blastocysts, which were subsequently transferred to pseudopregnant females to generate chimeric offspring. Male chimeras were bred with C57BI/6 female mice to produce heterozygotes. The germ line transmission was confirmed in the F1 generation using PCR genotyping strategy. Mice were then crossed to Flp-deletes mice to remove the neomycin cassette and thereafter crossed with Tie2CreER^{T2} animals to obtain endothelial cell-specific knock-out animals (Tie2-CreER^{T2};RIPK3^{IoxP/IoxP}=RIPK3^{ECKO}). A IoxP-PCR reaction was used for detection of the wt allele (+) and the flexed (fl) allele. To induce recombination, animals were treated with 5×1 mg/d tamoxifen (Sigma) and 7-9 days later experiments were started. RIPK3 deletion in endothelial cells was confirmed by comparing protein levels on isolated endothelial cells from lungs of knock-out animals (Tie2-CreER^{T2};RIPK3^{IoxP/IoxP}(−/−)) with endothelial cells from lungs of control animals (RIPK3^{rvfl} (+/+)) using Western blot. Quantification of in vivo permeability was performed using the Miles assay. Briefly, eleven days after induction of the knockout by tamoxifen, mice received a 100 μtail vein injection of 0.5% Evans blue dye in PBS. After 30 minutes mice were killed, and extravasated blue dye was eluted from the lungs with formamide at 56° C. and measured by spectrometry at 620 nm.

Human Samples: Frozen human tissue samples were obtained from Zyagen and stained for CD31 (Acris) and DR6 (Bioss). Experiments with human samples were performed according to the regulations of the local ethics committee of the Hessian Regional Medical Board, and informed consent was obtained from all subjects.

Example 2: Method to discriminate apoptotic from necrotic cell death

An assay to discriminate apoptotic from necroticinecroptotic cell death was established. To test the method HUVEC were used under control conditions or treated with various known stimuli for apoptosis (TRAIL and staurosporine) or necrosis (H₂O₂ and low oxygen concentrations). Cells were stained with EtDIII and Hoechst and nuclear morphology was analyzed. Control-treated cells showed kidney-like shaped nuclei with no signs of chromatin condensation or fragmentation and no EtDIII staining. In contrast, nuclei from apoptotic cells (TRAIL or staurosporine) were condensed and fragmented, some of which were also positive for EtDIII (Le. late apoptosis). Nuclei from necrotic cells (H₂O₂ or low oxygen concentrations) showed only minor changes in nuclear morphology without chromatin condensation or fragmentation but were positive for EtDIII (FIG. 2A). Results were validated by applying the same staining methods to mouse embryonic fibroblast L929 cells. These cells are commonly used as standard in vitro model system to study necroptosis, because stimulation of these cells with TNFα results in necroptotic cell death and can be blocked by the presence of the RIPK1 inhibitor Necrostatin-1 (Nec-1). L929 cells treated with INFα (necroptotic) showed a similar staining pattern as HUVECs treated with H₂O₂ or low oxygen, and this effect could be blocked by the addition of Nec-1 (FIG. 2B).

Example 3: Tumour cells induce necroptosis in endothelial cells in vitro

Based on the assay for to discriminating apoptotic from necroptotic cell death described herein above, it was shown that various tumour cells are able to induce endothelial cell necroptotic cell death (FIGS. 3A and 3B). Furthermore, the degree of endothelial cell necroptosis depends on the tumour cell number (FIG. 3C), i.e. the more tumour cells are added, the higher the percentage of endothelial cell necroptotic cell death. Applying the same method for visualising necrotidnecroptotic endothelial cells, it was shown that tumour cells are also able to induce endothelial cell death in vivo. Upon intravenous injection of tumour cells, endothelial cells of the mouse lung were positive for EtDIII without signs of nuclear condensation or fragmentation as visualized by DAPI staining. Furthermore, apoptotic markers such as cleaved caspase-3 could not be detected (FIGS. 3D). EtDIII-positive cells co-localised with ERG, a marker for endothelial cells (FIG. 3E).

Example 4: Increased tumour cell-induced endothelial cell death leads to increased metastasis

TGF-beta-activating kinase 1 (TAK1) is a signal molecule acting as molecular switch between cell survival and cell death. It was previously show that absence or inhibition of TAK1 results in increased necroptosis in endothelial cells (Morioka et al., 2012). Importantly, it was found herein that endothelial cells in which TAK1 was silenced using sRNA showed increased necroptosis upon stimulation with tumour cells (FIG. 4A).

Furthermore, mice that lack TAK1 specifically in endothelial cells (TAK^{e}n also showed increased necroptotic cell death in endothelial cells which coincided with increased tumour metastasis (FIGS. 4B to 4E).

Example 5: Pharmacological inhibition of tumour cell-induced endothelial cell death results in reduced metastasis formation

Endothelial cells treated with the necroptosis inhibitor necrostatin-1 (Nec-1) in vitro showed reduced tumour cell-induced necroptosis (FIG. 5A). Furthermore, animals treated with Nec-1 also showed reduced tumour cell-induced endothelial necroptosis and reduced metastasis formation in vivo (FIGS. 5A to 5E).

Example DR6 is required for tumour cell (TC) induced endothelial cell (EC) death and TC transmigration

An siRNA screen was performed to identify potential receptors that mediate tumour cell-induced endothelial cell necroptotic cell death. siRNAs specific for RIPK3 and TAK1 (MAP3K7) were used as a positive and negative control, respectively. The screen revealed that knockdown of DR6 (TNFRSF21) reduced endothelial cell necroptosis to an extent comparable to the reduction achieved by knockdown of RIPK3 (FIG. 6A). In order to confirm the data obtained in the siRNA screen with regard to DR6, different siRNAs specific for DR6 (siDR6#1, siDR6#2 and siDR6#3) were used to knock down DR6 in endothelial cells. This reduced the number of endothelial cells which died by necroptosis upon treatment with tumour cells (TCs) as compared to endothelial cells which had been treated with a control siRNA (siCTRL) (FIG. 6B). Knockdown of DR6 using the different siRNAs resulted in a reduction of DR6 expression by >70% (data not shown) and also reduced the transmigration of tumour cells through a HUVEC monolayer (FIG. 6C). Endothelial cell death and tumour cell transmigration were also reduced by a DR6-Fc fusion protein (DR6-Fc) in a concentration dependent manner. IgG1-Fc was used as a negative control (FIGS. 6D and 6E).

Example 7: Knockdown of RIPK3, MLKL and Caspase-8

RIPK3 and MLKL are considered to be specific regulators of necroptosis (Newton et al., 2014; Wang et al., 2014). Knockdown of RIPK3 or MLKL in endothelial cells in vitro blocked tumor cell-induced endothelial necroptotic cell death (FIG. 7A). In contrast, knockdown of caspase-8, one of the major negative regulators of necroptosis (Gunther et al., 2011; Oberst et al., 2011), resulted in an increase in tumor-cell induced endothelial necroptosis (Ag. 7A). Again, the degree of tumor cell-induced endothelial cell necroptosis correlated with the ability of tumor cells to migrate over an endothelial cell layer (compare FIG. 7A with FIG. 7B).

To further test whether loss of RIPK3 affects tumor cell-induced endothelial cell death, tumor cell extravasation and metastasis formation in vivo, endothelial cell-specific knock-out mice were generated by crossing tamoxifen-inducible Tie2-CreER^{T2} animals (Korhonen et al., 2009) with mice carrying foxed alleles of RIPK3 (Tie2-CreER^{T2};RIPK3^{IoxP/IoxP}, henceforth termed)RIPK3^{ECKO}). However, animals with endothelial cell-specific loss of RIPK3 showed reduced numbers of EthD-III-positive endothelial cells as well as reduced numbers of extravasated tumor cells 6 hours after tumor cell injection, and these animals developed fewer metastases (FIG. 7C-F).

Example 8: DR6 is expressed in different human organs

DR6, which was also found to be expressed in mouse and human endothelial cells of different vascular beds (FIG. 8), belongs to a subgroup of the TNF receptor family containing a cytosolic death domain that enables cell death signaling (Lavrik et al., 2005; Pan et al., 1998).

Example 9: Induction of necroptosis by DR6 ligand binding

Given that DR6 promotes tumor cell-induced endothelial necroptosis and metastasis, it was tested whether ligand binding to DR6 is required for these effects. To compete with the endogenous DR6 for a putative ligand, the DR6 ectodomain fused to an Fc fragment (DR6-Fc) was employed which functions as decoy receptor. Treatment of animals with DR6-Fc was sufficient to reduce endothelial necroptosis and tumor cell extravasation, and these animals also developed less metastases (FIG. 9A-D).

Example 10: Further functional characterization of the DR6 ligand

A previously identified ligand of DR6 is amyloid precursor protein (APP) (Nikolaev et al., 2009), that is highly expressed in the nervous system (Aydin et al., 2012; Muller and Zheng, 2012) as well as in several cell types including tumor cells (Hansel et al., 2003; Krause et al., 2008; Meng et al., 2001; Takagi et al., 2013; Woods and Padmanabhan, 2013) and can induce programmed cell death (Nikolaev et al., 2009). It was found that tumor cells in which APP mRNA expression levels were reduced to less than 3% using siRNA-mediated knockdown had lost the ability to induce endothelial necroptosis and showed a strongly reduced ability to transmigrate an endothelial cell layer when compared to tumor cells transfected with control-siRNA (FIG. 12A and B).

After intravenous injection of tumor cells with transiently reduced APP expression (FIG. 11A) into wild-type animals, we found significantly reduced numbers of necroptotic endothelial cells and extravasated tumor cells in the lungs of these animals (FIG. 12C-F). Importantly, tumor cells with strongly reduced APP expression showed normal proliferation, cell survival and basal migratory activity (FIG. 11B-D) but almost completely lost the ability to form metastases (FIG. 12C-F). This shows that APP expressed by tumor cells and endothelial DR6 are required for tumor cell-induced endothelial necroptosis and that this activity promotes tumor cell extravasation and metastasis formation.

### Further References

Cho, Y. S., Challa, S., Moquin, D., Genga, R., Ray, T. D., Guildford, M., and Chan, F. K. (2009). Phosphorylation-driven assembly of the RIP1-RIP3 complex regulates programmed necrosis and virus-induced inflammation. Cell 137, 1112-1123.

Dillon, C. P., Oberst, A., Weinlich, R., Janke, L. J., Kang, T. B., Ben-Moshe, T., Mak, T. W., Wallach, D., and Green, D. R. (2012). Survival function of the FADD-CASPASE-8-cFLIP(L) complex. Cell reports 1, 401-407.

Dillon, C. P., Weinlich, R., Rodriguez, D. A., Cripps, J.G., Quarato, G., Gurung, P., Verbist, K. C., Brewer, T. L., Llambi, F., Gong, Y. N., et al. (2014). RIPK1 Blocks Early Postnatal Lethality Mediated by Caspase-8 and RIPK3. Cell.

Heyder, C., Gloria-Maercker, E., Entschladen, F., Hatzmann, W., Niggemann, B., Zanker, K. S., and Dittmar, T. (2002). Realtime visualisation of tumor cell/endothelial cell interactions during transmigration across the endothelial barrier. Journal of cancer research and clinical oncology 128, 533-538.

Joyce, J. A., and Pollard, J. W. (2009). Microenvironmental regulation of metastasis. Nature reviews Cancer 9, 239-252.

Kebers, F., Lewalle, J. M., Desreux, J., Munaut, C., Devy, L., Foidart, J. M., and Noel, A. (1998). Induction of endothelial cell apoptosis by solid tumor cells. Experimental cell research 240, 197-205.

Labelle, M., and Hynes, R. O. (2012). The initial hours of metastasis: the importance of cooperative host-tumor cell interactions during hematogenous dissemination. Cancer discovery 2, 1091-1099.

Lin, J., Li, H., Yang, M., Ren, J., Huang, Z., Han, F., Huang, J., Ma, J., Zhang, D., Zhang, Z., et al. (2013). A role of RIP3-mediated macrophage necrosis in atherosclerosis development. Cell reports 3, 200-210.

Lin, R. Z., Wang, T. P., Hung, R. J., Chuang, Y. J., Chien, C. C., and Chang, H. Y. (2011). Tumor-induced endothelial cell apoptosis: roles of NAD(P)H oxidase-derived reactive oxygen species. Journal of cellular physiology 226, 1750-1762.

Linkermann, A., Brasen, J. H., Himmerkus, N., Liu, S., Huber, T. B., Kunzendorf, U., and Krautwald, S. (2012). Rip1 (receptor-interacting protein kinase 1) mediates necroptosis and contributes to renal ischemia/reperfusion injury. Kidney international 81, 751-761.

Linkermann, A., and Green, D. R. (2014). Necroptosis. The New England journal of medicine 370, 455-465.

Mierke, C. T. (2008). Role of the endothelium during tumor cell metastasis: is the endothelium a barrier or a promoter for cell invasion and metastasis? Journal of biophysics 2008, 183516.

Morioka, S., Inagaki, M., Komatsu, Y., Mishina, Y., Matsumoto, K., and Ninomiya-Tsuji, J. (2012). TAK1 kinase signaling regulates embryonic angiogenesis by modulating endothelial cell survival and migration. Blood 120, 3846-3857.

Murphy, J. M., and Silke, J. (2014). Ars Moriendi; the art of dying well - new insights into the molecular pathways of necroptotic cell death. EMBO reports 15, 155-164.

Reymond, N., d′Agua, B.B., and Ridley, A.J. (2013). Crossing the endothelial barrier during metastasis. Nature reviews Cancer 13, 858-870.

Sawai, H., and Domae, N. (2011). Discrimination between primary necrosis and apoptosis by necrostatin-1 in Annexin V-positive/propidium iodide-negative cells. Biochemical and biophysical research communications 411, 569-573.

Smith, C. C., Davidson, S. M., Lim, S. Y., Simpkin, J. C., Hothersall, J. S., and Yelton, D. M. (2007). Necrostatin: a potentially novel cardioprotective agent? Cardiovascular drugs and therapy/sponsored by the International Society of Cardiovascular Pharmacotherapy 21, 227-233.

Aydin, D., Weyer, S. W., and Muller, U. C. (2012). Functions of the APP gene family in the nervous system: insights from mouse models. Experimental brain research 217, 423-434.

Beisner, D. R., Ch'en, I. L., Kolla, R. V., Hoffmann, A., and Hedrick, S. M. (2005). Cutting edge: innate immunity conferred by B cells is regulated by caspase-8. Journal of immunology 175, 3469-3473.

Gunther, C., Martini, E., Wittkopf, N., Amann, K., Weigmann, B., Neumann, H., Waldner, M. J., Hedrick, S. M., Tenzer, S., Neurath, M. F., et al. (2011). Caspase-8 regulates TNF-alpha-induced epithelial necroptosis and terminal ileitis. Nature 477, 335-339.

Hansel, D. E., Rahman, A., Wehner, S., Herzog, V., Yeo, C. J., and Maitra, A. (2003). Increased expression and processing of the Alzheimer amyloid precursor protein in pancreatic cancer may influence cellular proliferation. Cancer research 63, 7032-7037.

Korhonen, H., Fisslthaler, B., Moers, A., Wirth, A., Habermehl, D., Wieland, T., Schutz, G., Wettschureck, N., Fleming, I., and Offermanns, S. (2009). Anaphylactic shock depends on endothelial Gq/G11. The Journal of experimental medicine 206, 411-420.

Krause, K., Karger, S., Sheu, S.Y., Aigner, T., Kursawe, R., Gimm, 0., Schmid, K. W., Dralle, H., and Fuhrer, D. (2008). Evidence for a role of the amyloid precursor protein in thyroid carcinogenesis. The Journal of endocrinology 198, 291-299.

Lavrik, I., Golks, A., and Krammer, P. H. (2005). Death receptor signaling. Journal of cell science 118, 265-267.

Liang, C. C., Park, A. Y., and Guan, J. L. (2007). In vitro scratch assay: a convenient and inexpensive method for analysis of cell migration in vitro. Nature protocols 2, 329-333.

Meng, J. Y., Kataoka, H., Itoh, H., and Koono, M. (2001). Amyloid beta protein precursor is involved in the growth of human colon carcinoma cell in vitro and in vivo. International journal of cancer Journal international du cancer 92, 31-39.

Muller, U. C., and Zheng, H. (2012). Physiological functions of APP family proteins. Cold

Spring Harbor perspectives in medicine 2, a006288.

Newton, K., Dugger, D. L., Wickliffe, K. E., Kapoor, N., de Almagro, M. C., Vucic, D., Komuves, L., Ferrando, R. E., French, D. M., Webster, J., et al. (2014). Activity of protein kinase RIPK3 determines whether cells die by necroptosis or apoptosis. Science 343, 1357-1360.

Nikolaev, A., McLaughlin, T., O'Leary, D. D., and Tessier-Lavigne, M. (2009). APP binds DR6 to trigger axon pruning and neuron death via distinct caspases. Nature 457, 981-989.

Oberst, A., Dillon, C. P., Weinlich, R., McCormick, L. L., Fitzgerald, P., Pop, C., Hakem, R.,

Salvesen, G. S., and Green, D. R. (2011). Catalytic activity of the caspase-8-FLIP(L) complex inhibits RIPK3-dependent necrosis. Nature 471, 363-367.

Pan, G., Bauer, J. H., Haridas, V., Wang, S., Liu, D., Yu, G., Vincenz, C., Aggarwal, B. B., Ni, J., and Dixit, V. M. (1998). Identification and functional characterization of DR6, a novel death domain-containing TNF receptor. FEBS letters 431, 351-356.

Schumacher, D., Strilic, B., Sivaraj, K. K., Wettschureck, N., and Offermanns, S. (2013). Platelet-derived nucleotides promote tumor-cell transendothelial migration and metastasis via P2Y2 receptor. Cancer cell 24, 130-137.

Sivaraj, K. K., Takefuji, M., Schmidt, I., Adams, R. H., Offermanns, S., and Wettschureck, N. (2013). G13 controls angiogenesis through regulation of VEGFR-2 expression. Developmental cell 25, 427-434.

Takagi, K., Ito, S., Miyazaki, T., Miki, Y., Shibahara, Y., Ishida, T., Watanabe, M., Inoue, S., Sasano, H., and Suzuki, T. (2013). Amyloid precursor protein in human breast cancer: an androgen-induced gene associated with cell proliferation. Cancer science 104, 1532-1538.

Wang, H., Sun, L., Su, L., Rizo, J., Liu, L., Wang, L. F., Wang, F. S., and Wang, X. (2014).

Mixed lineage kinase domain-like protein MLKL causes necrotic membrane disruption upon phosphorylation by RIP3. Molecular cell 54, 133-146.

Woods, N. K., and Padmanabhan, J. (2013). Inhibition of amyloid precursor protein processing enhances gemcitabine-mediated cytotoxicity in pancreatic cancer cells. The

Journal of biological chemistry 288, 30114-30124.

Young-Pearse, T. L., Bai, J., Chang, R., Zheng, J.B., LoTurco, J. J., and Selkoe, D. J. (2007). A critical function for beta-amyloid precursor protein in neuronal migration revealed by in utero RNA interference. The Journal of neuroscience : the official journal of the Society for

Neuroscience 27, 14459-14469.

1. (canceled)

2. A method of preventing the metastasis of tumours by inhibiting necroptosis comprising administering a pharmaceutically effective amount of an inhibitor of necroptosis to a subject in need thereof.

3. A method for modulating the transmigration of metastasising tumour cells through endothelium by modulating necroptosis, comprising the steps of:
a) contacting endothelium with a modulator of necroptosis; and
b) providing metastasising tumour cells and allowing said metastasising tumour cells to transmigrate through the endothelium.

4. An in vitro method of identifying an inhibitor of necroptosis suitable as a lead compound and/or as a medicament for the prevention of tumour metastasis, comprising the steps of:
a) allowing metastasizing tumour cells to transmigrate the endothelium
i) in the presence of a test agent and
ii) in the absence of said test agent;
b) determining the level of tumour cell transmigration through the endothelium and/or the level of endothelial cell necroptosis for a)i) and a)ii);
c) comparing the level(s) determined in step b) for a)i) with the level(s) determined in step b) for a)ii),
wherein a decrease in the level(s) for a)i) as compared to the level(s) of a)ii) is indicative for the test agent to be an inhibitor of necroptosis suitable as a lead compound and/or as a medicament for the prevention of tumour metastasis.

5. An inhibitor or modulator of necroptosis that is selected from the group consisting of an inhibitor of receptor interacting protein 1 (RIPK1), an inhibitor of receptor interacting prtein 3 (RIPK3), an inhibitor of mixed lineage kinase domain like protein (MLKL) or a combination thereof.

6. The methods of claim 2, wherein the subject or the endothelium is mammalian.

7. The inhibitor or modulator according to claim 5, wherein the inhibitor or modulator prevents or moduklates the formation of a necrosome andor a necroptosis-inducing activity of the necrosome.

8. The inhibitor or modulator according to claim 5, wherein the inhibitor or modulator is an antibody, an antibody mimetic, a dominant negative protein, a siRNA, a shRNA, a miRNA, a ribozyme, an aptamer, anucleic acid molecule, an antisense nucleic acid molecule, a small molecue or modified version of these.

9. The inhibitor or modulator accrding to claim 5, wherein the inhibitor or modulator is an inhibitor of RIPK3.

10. The inhibitor or modulator according to claim 5, wherein the inhibitor or the modulator is selected from the group consisting of necrostatin-1 (Nec-1; 5-(1H-indol-3-ylmethyl)-3 -methyl-2-thioxo-4-imidazolidinone, 5-iIndo1-3 -ylmethyl)-3 -methyl-2-thio-hydantoin), necrostatin-1 stable (5- ((7-chloro -1H-indo1-3 -yl)methyl)-3 -methyl-2,4-imidazolidinedione, 5- ((7-chloro- 1H-indo1-3 - yl)methyl)-3 -methylimidazolidine-2,4-dione) , necrostatin-1 inactive (5- ((1H-indo1-3 -yl)methyl)-2-thioxoimidazolidin-4-one), Necrosulfonamide (NSA; (E)-N-(4-(N-(3-methoxypyrazin-2-yl)sulfamoyl)phenyl)-3-(5-nitrothiophene-2-yl)acrylamide), an anti-RIPK3 siRNA, an anti-MLKL siRNA or a combination thereof.

11. The method of claim 3,wherein the modulator is an inhibitor of TGF-beta-activating kinase 1 (TAK1).

12. The inhibitor or modulator acccording to claim 5, that is an inhibitor of death receptor 6 (DR6).

13. The inhibitor or modulator according to claim 5 having admixed thereto or being associated in a separate container with a further pharmaceutically active agent.

14. A method of identifying necroptotic and necrotic cells comprising:
a) contacting cells with a marker for plasma membrane breakdown;
b) contacting said cells with a marker for chromatin condensation and/or chromatin fragmentation; and
c) determining whether plasma membrane breakdown and chromatin condensation and/or chromatin fragmentation occurs, wherein it is indicative that said cells are necroptotic or necrotic, if plasma membrane breakdown is determined in step c) in the absence of chromatin condensation and/or chromatin fragmentation as occurring in apoptotic cells.

15. The method of claim 14, wherein the marker for plasma membrane breakdown in step a) is selected from the group consisting of a membrane-impermeable DNA-binding dye and/or the marker for chromatin condensation and/or chromatin fragmentation is selected from the group consisting of membrane-permeable DNA-binding dyes.

16. The method of claim 2, wherein the inhibitor or modulator is selected from the group consisting of an inhibitor of receptor interacting protein 1 (RIPK1), an inhibitor of receptor interacting protein 3 (RIPK3), an inhibitor of mixed lineage kinase domain like protein (MLKL) or a combination thereof.

17. The method of claim 2, wherein the inhibitor or modulator is selected from the group consisting of necrostatin-1 (Nec-1; 5-(1H-indol-3-ylmethyl)-3-methyl-2-thioxo-4-imidazolidinone, 5-iIndo1-3-ylmethyl)-3-methyl-2-thio-hydantoin), necrostatin-1 stable (5-((7-chloro-1H-indol-3-yl)methyl)-3-methyl-2,4-imidazolidinedione, 5-((7-chloro-1H-indol-3-yl)methyl)-3-methylimidazolidine-2,4-dione), necrostatin-1 inactive (5-((1H-indol-3-yl)methyl)-2-thioxoimidazolidin-4-one), Necro sulfonamide (NSA; (E)-N-(4-(N-(3-methoxypyrazin-2-yl)sulfamoyl)phenyl)-3-(5-nitrothiophene-2- yl)acrylamide), an anti-RIPK3 siRNA, an anti-MLKL siRNA or a combination thereof.

18. The method of claim 3, wherein the inhibitor or modulator is selected from the group consisting of an inhibitor of receptor interacting protein 1 (RIPK1), an inhibitor of receptor interacting protein 3 (RIPK3), an inhibitor of mixed lineage kinase domain like protein (MLKL) or a combination thereof.

19. The method of claim 3, wherein the inhibitor or the modulator is selected from the group consisting of necrostatin-1 (Nec-1; 5-(1H-indol-3-ylmethyl)-3-methyl-2-thioxo-4-imidazolidinone, 5-iIndol-3-ylmethyl)-3-methyl-2-thio-hydantoin), necrostatin-1 stable (5-((7-chloro-1H-indol-3-yl)methyl)-3-methyl-2,4-imidazolidinedione, 5-((7-chloro- 1H-indol-3-yl)methyl)-3-methylimidazolidine-2,4-dione), necrostatin-1 inactive (5-((1H-indol-3-yl)methyl)-2-thioxoimidazolidin-4-one), Necrosulfonamide (NSA; (E)-N-(4-(N-(3 -methoxypyrazin-2-yl)sulfamoyl)phenyl)-3-(5-nitrothiophene-2-yl)acrylamide), an anti-RIPK3 siRNA, an anti-MLKL siRNA or a combination thereof.
